(19) United States
(12) Patent Application Publication
(10) Pub. No.: US 2026/0207508 A1
(43) Pub. Date: Jul. 23, 2026

(54) CYCLODEXTRIN-POLY (BETA AMINO ESTER) MACROMOLECULES FOR NANOPARTICLE DRUG DELIVERY

(71) Applicants: UNIVERSITY OF MASSACHUSETTS, Westborough, MA (US); BOARD OF REGENTS OF THE UNIVERSITY OF TEXAS SYSTEM, Austin, TX (US)

(72) Inventors: Rachael W. Sirianni, Princeton, MA (US); Sauradip Chaudhuri, Framingham, MA (US)

(21) Appl. No.: 19/144,113

(22) PCT Filed: Jan. 19, 2024
(86) PCT No.: PCT/US2024/012152

§ 371 Date: Jun. 27, 2025

Related U.S. Application Data
(60) Provisional application No. 63/440,026, filed on Jan. 19, 2023.

(51) Int. Cl. A61K 9/16; A61K 31/4045; A61K 47/60; A61K 47/61; A61K 47/69

(52) U.S. Cl. CPC A61K 9/1652; A61K 31/4045; A61K 47/60; A61K 47/61; A61K 47/6941

(57) ABSTRACT

Compositions that facilitate therapeutic delivery, as well as methods of making and using the same, are described herein. In particular, the composition including a cross-linked network of cyclic macromolecules and a plurality of functional groups, wherein the cyclic macromolecules are covalently cross-linked to one another by a plurality of cross-linking agents, and the plurality of functional groups are covalently associated with the cross-linked network of cyclic macromolecules, wherein the plurality of functional groups include (i) a proximal end covalently associated with the cross-linked network of cyclic macromolecules, and (ii) a distal end protruding out of the cross-linked network of cyclic macromolecules, wherein the distal end comprises a charge-imparting group.

### RELATED APPLICATIONS

This application claims priority to U.S. Provisional App. 63/440,026, filed 19 Jan. 2023 and titled CYCLODEXTRIN-POLY (BETA AMINO ESTER) MACROMOLECULES FOR NANOPARTICLE DRUG DELIVERY. This application is related to U.S. patent application Ser. No. 17/613,197, filed on Nov. 22, 2021, which is a U.S. National Stage Application of PCT/US2020/024627, filed on Mar. 25, 2020, which claims priority to U.S. Provisional Patent Application No. 62/850,308, filed on May 20, 2019. This application is also related to EPO patent application No. 20810786.2, filed on Nov. 8, 2021, which is a European National Stage Application of PCT/US2020/024627. The entirety of each of the aforementioned applications is incorporated herein by reference.

### STATEMENT REGARDING FEDERALLY SPONSORED RESEARCH

This invention was made with government support under HD099543 awarded by The National Institutes of Health. The government has certain rights in the invention.

### BACKGROUND

The administration of free drugs suffer from numerous limitations, such as solubility and limited pharmacokinetics. Current drug delivery agents (e.g., agents that promote drug delivery, such as excipients and/or particle systems) also suffer from numerous limitations, such as poor stability, limited drug loading capacities, limited abilities for sustained drug release and distribution, inefficient fabrication methods, limited biological stability, aggregation, disassembly, and sub-optimal biophysical properties for in vivo applications. Various embodiments of the present disclosure address the aforementioned limitations.

### SUMMARY

The present disclosure described compositions that facilitate therapeutic delivery, as well as methods of making and using the same. An aspect of the present disclosure relates to a composition comprising a cross-linked network of cyclic macromolecules and a plurality of functional groups, wherein the cyclic macromolecules are covalently cross-linked to one another by a plurality of cross-linking agents, and the plurality of functional groups are covalently associated with the cross-linked network of cyclic macromolecules, wherein the plurality of functional groups comprise (i) a proximal end covalently associated with the cross-linked network of cyclic macromolecules, and (ii) a distal end protruding out of the cross-linked network of cyclic macromolecules, wherein the distal end comprises a charge-imparting group.

In any aspect or embodiment described herein, the charge-imparting group is positioned at the distal ends of the plurality of functional groups.

In any aspect or embodiment described herein, the charge-imparting group is selected from the group consisting of proton donating groups, proton accepting groups, positively charged groups, negatively charged groups, hydrogen donating groups, amine groups, N,N-dimethylethylamine, carboxylic acids, phosphates, sulfonates, and combinations thereof.

In any aspect or embodiment described herein, the charge-imparting group is a negatively charged group.

In any aspect or embodiment described herein, the charge-imparting group comprises carboxylic acids.

In any aspect or embodiment described herein, the plurality of functional groups are covalently coupled directly to at least some of the cyclic macromolecules.

In any aspect or embodiment described herein, the plurality of functional groups are covalently coupled directly to at least some of the cross-linking agents.

In any aspect or embodiment described herein, the plurality of functional groups comprises functional groups with a molecular weight of less than 500 Da or a molecular weight of less than 300 Da.

In any aspect or embodiment described herein, the composition is in the form of particles.

In any aspect or embodiment described herein, the particles are in a aggregated form (e.g., a depot or depot formulation).

In any aspect or embodiment described herein, the particles are in non-aggregated form.

In any aspect or embodiment described herein, the particles comprise an amphiphilic core and a hydrophilic outer surface, wherein the hydrophilic outer surface has a negative or neutral charge.

In any aspect or embodiment described herein, the particles comprise diameters ranging from 100 nm to about 500 nm.

In any aspect or embodiment described herein, the molar ratio of the cyclic macromolecules and the cross-linking agents in the composition is 1:15 to 1:50 (e.g., 1:20 to 1:50, 1:20 to 1:25, 1:22, or 1:45).

In any aspect or embodiment described herein, the molar ratio of the cross-linking agents to the amine-based functional groups in the composition is 1:0.5 to 1:1.75 to (e.g., 1:0.6 to 1:1.6, 1:0.7 to 1:1.5, 1:1.5 or 1.4:1).

In any aspect or embodiment described herein, the cyclic macromolecules are selected from the group consisting of cyclic oligosaccharides, macrocycles, cyclodextrins, and combinations thereof.

In any aspect or embodiment described herein, the cross-linking agents comprise polyacrylic acids.

In any aspect or embodiment described herein, the cyclic macromolecules comprise β-cyclodextrin.

In any aspect or embodiment described herein, the functional groups comprise or are polymers, polyethylene glycol, alkyl chains, amine-based functional groups, or a combination thereof.

In any aspect or embodiment described herein, the functional groups comprise amine-based functional groups, wherein the amine-based functional groups are exposed to a surface of the particles.

In any aspect or embodiment described herein, the functional groups comprise polyethylene glycol functional groups.

In any aspect or embodiment described herein, the functional groups comprise polyethylene glycol functional groups that comprise a chain of at least ten atoms

In any aspect or embodiment described herein, the cross-linking agents and the functional groups form a polymer network.

In any aspect or embodiment described herein, the polymer network comprises poly (β-amino ester).

In any aspect or embodiment described herein, the composition further comprises an active agent.

In any aspect or embodiment described herein, the active agent is associated with the composition through non-covalent interactions.

In any aspect or embodiment described herein, the active agent is ionized.

In any aspect or embodiment described herein, the active agent is a hydrophobic molecule.

In any aspect or embodiment described herein, the active agent constitutes at least about 0.1% by weight of the composition.

In any aspect or embodiment described herein, the active agent includes or is a small molecule, peptide, drug, hormone, analgesic, anti-epileptic, chemotherapeutic, neuroprotective agent, anti-inflammatory agent, anti-neuro-inflammatory agent, cytotoxic agent, histone deacetylase inhibitor, proteasome inhibitor, imaging agent, targeting agent, nucleotides, therapeutic nucleic acid, deoxyribonucleic acid (DNA), ribonucleic acid (RNA), messenger RNA (mRNA), microRNA (miRNA), small interfering RNA (siRNA), short hairpin RNA (shRNA), antisense RNA, circular RNA (circRNA), ribozyme (RNAzyme), deoxyribozymes (DNAzyme), or combinations thereof.

A Further aspect of the present disclosure relates to a method of administering an active agent to a subject, the method comprising administering a composition of the present disclosure to the subject, wherein the composition is associated with the active agent.

In any aspect or embodiment described herein, the active agent is associated with the composition through non-covalent interactions.

In any aspect or embodiment described herein, the active agent has IC₅₀ values of less than <1.0 μM.

In any aspect or embodiment described herein, the composition enhances the spatial delivery of the active agent to a desired region or tissue when compared to the administration of the active agent without the composition.

In any aspect or embodiment described herein, administering the composition is intrathecal-cisterna magna (IT-CM) administration for delivery of the active agent to a brain (e.g., brain subarachnoid space) of the subject.

In any aspect or embodiment described herein, administering the composition is intrathecal-lumbar (IT-L) administration for delivery of the active agent to a spinal cord of the subject.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating embodiments of the disclosure and are not to be construed as limiting the disclosure. Further objects, features, and advantages of the disclosure will become apparent from the following detailed description taken in conjunction with the accompanying figures showing illustrative embodiments of the disclosure.

FIG. 1A provides an illustration of a composition with a cross-linked network of cyclic macromolecules according to an aspect of the present disclosure.

FIG. 1B illustrates a method for administration and sustained release of an active agent by utilizing the compositions of the present disclosure.

FIG. 2 provides the structure of Panobinostat.

FIG. 3 provides the structure of 2-hydroxypropyl-β-cyclodextrin.

FIG. 4 illustrates an exemplary method of synthesizing of cyclodextrin nanoparticles (CDNs).

FIG. 5A shows exemplary functional groups and cross-linkers, including those utilized in CDN-3, CDN-4, and CDN-5.

FIG. 5B shows the functional groups and cross-linkers of CDN-3, CDN-4, and CDN-5, as well as maximum panobinostat loading (%) and zeta potential (ZP) in mV.

FIG. 6 illustrates an exemplary schematic of the preparation of the panobinostat loaded cyclodextrin nanoparticle (CDN).

FIG. 7 illustrates an exemplary plate for determining panobinostat loaded in cyclodextrin nanoparticles (CDNs), including a standard calibration curve and a sample, each performed in triplicate.

FIG. 8 provides an exemplary calibration curve for determining Panobinostat loaded cyclodextrin nanoparticles (CDNs).

FIG. 9A provides an illustration of a panobinostat loaded CDN-3.

FIG. 9B show the observed stability of panobinostat loaded CDN-3 (Pb-CDN-3) at days 0, 2, 4, and 10.

FIG. 9C show the observed stability of panobinostat loaded CDN-3 (Pb-CDN-3) at days 0, 2, 4, and 10.

FIG. 9D provides an illustration of a CDN-4 and panobinostat under varying pHs.

FIG. 9E shows the observed stability of panobinostat loaded CDN-4 (Pb-CDN-4) at days 0, 1, and 5.

FIG. 9F shows the stability of pCD, pCDN-3, pCDN-4, and pCDN-5 at pH 4.0, 7.4, and 10.2.

FIG. 10A provides an illustration of the structure of CDN-5.

FIG. 10B show the observed stability of panobinostat loaded CDN-5 (Pb-CDN-5) at days 0, 3, 6, and 12 for 5.1 with 23.7% loading, 5.2 with 18.1% loading, and 5.4 with 15.6% loading.

FIG. 10C show the observed stability of panobinostat loaded CDN-5 (Pb-CDN-5) at days 0, 3, 6, and 12 for 5.1 with 23.7% loading, 5.2 with 18.1% loading, and 5.4 with 15.6% loading

FIG. 10D show the observed stability of 40% th loaded panobinostat CDN-5 (Pb-CDN-5) at days 0, 3, 6, and 12.

FIG. 10E show the observed stability of 40% th loaded panobinostat CDN-5 (Pb-CDN-5) at days 0, 3, 6, and 12.

FIG. 11A demonstrates that the exemplary panobinostat loaded CDN-5 nanoparticles are able to target the spinal cord.

FIG. 11B demonstrates that assembled particles transited the subarachnoid space and that particles must be assembled to transit the subarachnoid space.

FIG. 11C illustrates that IT-CM (intrathecal-cisterna magna) administration is a preferred route of administration over IT-L (intrathecal-lumbar) for achieving delivery to the brain subarachnoid space, and IT-L (intrathecal-lumbar) administration is a preferred route of administration over IT-CM (intrathecal-cisterna magna) for achieving delivery to the spinal cord.

FIG. 12A shows representative images of payload delivery to cervical lymphatics following administration of IR780-loaded CDNs into the cisterna magna of healthy mice.

FIG. 12B shows representative images of payload (IR780) delivery to the neuroaxis following administration of IR780-loaded CDNs into the cisterna magna of healthy mice at an early time point (top) and late time point (bottom).

FIGS. 13A, 13B, 13C, and 13D demonstrate that the payload (panobinostat) activates pharmacodynamic targets, thereby resulting in increased levels of acetylation of lysine 9 on histone H3 (H3K9ac), when administered intrathecally or intraperitoneally.

FIG. 14 shows representative matrix-assisted laser desorption ionization mass spectroscopy imaging (MALDI-MSI) data quantifying the concentration of panobinostat in tissue slices obtained from female C57BL6 mice that received an infusion of either panobinostat loaded into CDN-5 (pCDN) or panobinostat solubilized in beta-cyclodextrin (pCD). These data visually demonstrate enhancements in tissue penetration in periventricular brain tissue when panobinostat is delivered from the nanoparticle system.

FIG. 15 provides quantitative measurement of panobinostat levels at different regions of the spinal cord. Tissues were obtained as described for FIG. 14. These data quantitatively demonstrate improvements in the rostral-caudal distribution of panobinostat when the drug is delivered from the nanoparticle system.

FIG. 16 provides quantitative measurement of panobinostat concentration in different brain regions when it is administered by intravenous infusion. FVB mice received 10 mg/kg panobinostat that was freely solubilized in 0.8% dimethyl sulfoxide (DMSO), 0.8% Tween® 80, and 19.2% polyethylene glycol 300 (PEG300). The brains were dissected into major regions and homogenized for subsequent measurement of drug levels with liquid chromatography-mass spectrometry (LC-MS). These data provide reference values for panobinostat distribution in the brain.

FIG. 17. Shows representative matrix-assisted laser desorption ionization mass spectroscopy imaging (MALDI-MSI) data quantifying the concentration of panobinostat in tissue slices obtained from female C57BL6 mice that received an infusion of either panobinostat loaded into CDN-5 (pCDN) or panobinostat solubilized in beta cyclodextrin (pCD). These data visually demonstrate enhancements in tissue penetration in the spinal cord when panobinostat is delivered from the nanoparticle system.

FIG. 18 shows the concentration of panobinostat after 2 hours for the entire brain (“brain”), the peri-ventricular tissue (“ventricular”), cervical spinal cord (“SC-C”), thoracic spinal cord (“SC-T”), and the lumbar spinal cord (“SC-L”). FIG. 19 shows aggregate data from multiple studies and demonstrates that pCDNs enhance drug delivery relative to pCDs in both healthy and tumor-bearing mice.

FIG. 19 shows a delivery ratio (concentration of panobinostat as delivered from pCDN divided by the concentration of panobinostat as delivered by pCD) in the entire brain (“brain”), peri-ventricular tissue (“ventricle”), the tumor as identified on serial hematoxylin and eosin (H&E) stained sections (tumor), and a representative section of the spinal cord containing tumor as identified by hematoxylin and eosin (H&E) staining (“spinal cord”). “X” refers to “not detected” (i.e., 0, which cannot be plotted on a log scale). FIG. 20 shows aggregate data from multiple studies and demonstrates that pCDNs enhance delivery relative to pCDs in both healthy and tumor-bearing mice.

FIG. 20A, 20B, 20C, 20D, and 20E shows the data from three replicates of NOD scid gamma (NSG) mice bearing orthotopic, patient derived medulloblastoma xenografts treated with various doses of panobinostat loaded in CDN-5 (pCDN-5) or non-drug loaded CDN-5 (i.e., blank CDN-5 or bCDNs) through cisterna magna (ICM) or intratumoral (IT) administration, wherein FIG. 20A showing the data from Experiment 1, FIGS. 20B and 20C show the data from Experiment 2, and FIGS. 20D and 20E show the data from Experiment 3.

FIG. 21 shows the percent release of panobinostat from panobinostat loaded in CDN-5 (pCDN-5). These data demonstrate that the release of panobinostat from pCDN-5 is sustained.

### DETAILED DESCRIPTION

It is to be understood that both the foregoing general description and the following detailed description are illustrative and explanatory and are not restrictive of the subject matter, as claimed. The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value in the range, to the tenth of the unit of the lower limit unless the context clearly dictates otherwise (for example, in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either/or both of those included limits are also included in the disclosure.

It should also be understood that, in certain methods or processes described herein that include more than one step or act, the order of the steps or acts of the method is not necessarily limited to the order in which the steps or acts of the method are recited unless the context indicates otherwise.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (that is, to at least one or one or more of) of the grammatical object of the article unless the context clearly indicates otherwise. By way of example, “an element” or “component” means one element/components or more than one element/component, unless otherwise indicated.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, that is, “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (that is, “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended (that is, to mean including but not limited to). It is expressly contemplated that all embodiments, and claims reciting one of the open-ended transitional phrases can be written with any other transitional phrase, which may be more limiting, unless clearly precluded by the context or art. Only the transitional phrases “consisting of” and “consisting essentially of” shall be closed or semi-closed transitional phrases, respectively, as set forth in the United States Patent Office Manual of Patent Examining Procedures, Section 2111.03.

The term “administration of” and “administering”, as used herein, refers to giving, providing, applying, or dispensing the composition, for example, of the present disclosure, by any suitable route. In this disclosure suitable routes of administration include oral, intravenous, intramuscular, subcutaneous, inhaled, buccal, transmucosal, and intranasal administration.

The terms “effective,” “therapeutically effective,” “effective amount/dose,” “pharmaceutically effective amount/dose,” “pharmaceutically effective amount/dose,” or and “therapeutically effective amount/dose” are used to describe an amount/dose of a compound or composition which, when used within the context of its intended use, and either in a single dose or, more preferably after multiple doses within the context of a treatment regimen, effects an intended result, such as an improvement in or prevention of a disease, disorder, or condition, or amelioration (for example, alleviate to some extent, preferably all) or reduction in one or more symptoms associated with a disease, disorder, or condition. The terms “effective” and “therapeutically effective” subsume all other “effective amount” or “effective concentration” terms, which are otherwise described or used in the present application. The effective amount depends on the type of disease, the composition used, the route of administration, the type of mammal being treated, the physical characteristics of the specific mammal under consideration, concurrent medication, and other factors which those skilled in the medical arts will recognize.

Current drug delivery agents suffer from numerous limitations. Such limitations include poor stability, limited drug loading capacities, limited abilities for sustained release and distribution, and inefficient fabrication methods. Additional limitations include limited biological stability, aggregation, disassembly, and sub-optimal biophysical properties for in vivo applications. Various embodiments of the present disclosure address the aforementioned limitations.

An aspect of the present disclosure pertains to compositions that include one or more of the following components: cyclic macromolecules, cross-linking agents, functional groups, and active agents. In some embodiments, the compositions of the present disclosure include a cross-linked network of cyclic macromolecules. In some embodiments, the cyclic macromolecules are covalently cross-linked to one another by a plurality of cross-linking agents.

In some embodiments, the compositions of the present disclosure also include a plurality of functional groups. In some embodiments, the plurality of functional groups are covalently associated with the cross-linked network of cyclic macromolecules. In some embodiments, the plurality of functional groups include a proximal end that is covalently associated with the cross-linked network of cyclic macromolecules, and a distal end that protrudes out of the cross-linked network of cyclic macromolecules. In any aspect or embodiment described herein, the distal end includes a charge-imparting group.

Thus, an aspect of the present disclosure relates to a composition comprising a cross-linked network of cyclic macromolecules and a plurality of functional groups, wherein the cyclic macromolecules are covalently cross-linked to one another by a plurality of cross-linking agents, and the plurality of functional groups are covalently associated with the cross-linked network of cyclic macromolecules, wherein the plurality of functional groups comprise (i) a proximal end covalently associated with the cross-linked network of cyclic macromolecules, and (ii) a distal end protruding out of the cross-linked network of cyclic macromolecules, wherein the distal end comprises a charge-imparting group.

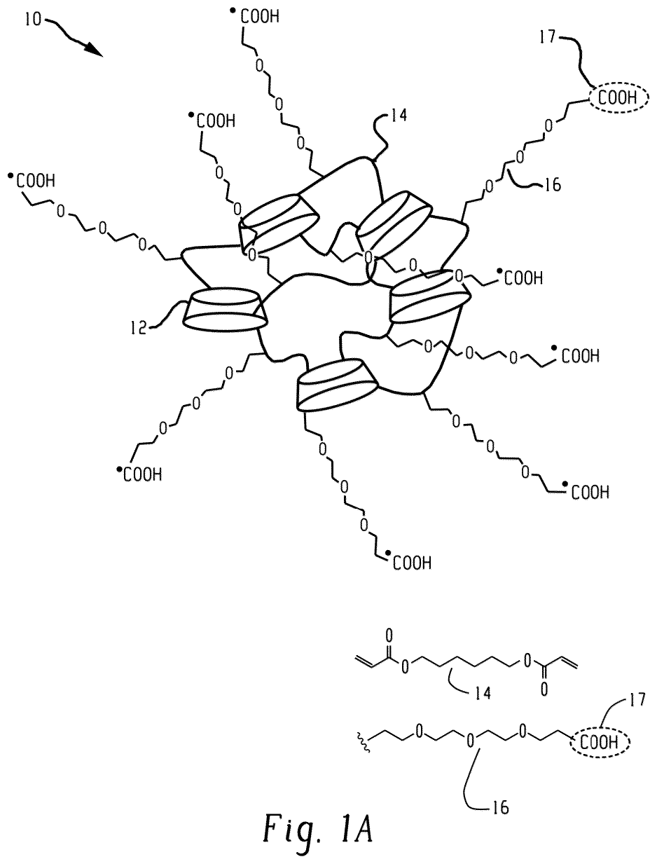

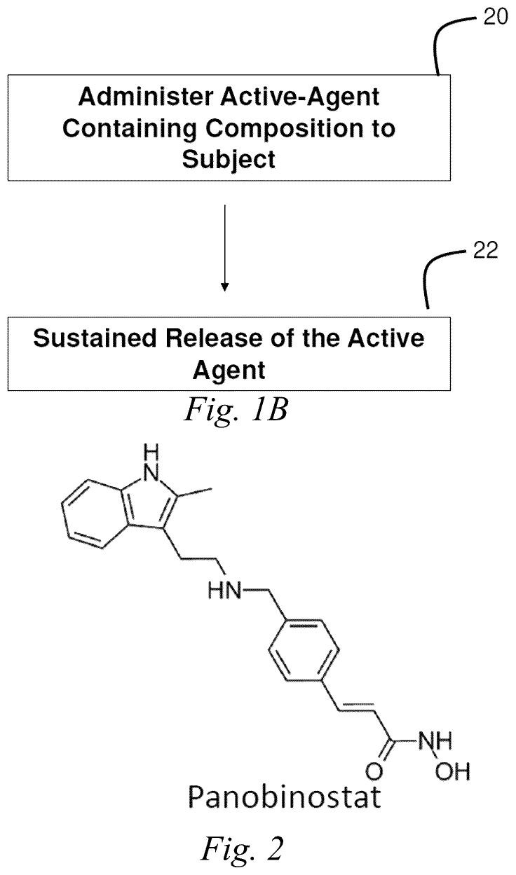

An example of a composition of the present disclosure is illustrated in FIG. 1A as composition 10. In this example, composition 10 includes a covalently cross-linked network of cyclic macromolecules 12 that are cross-linked to one another by a plurality of cross-linking agents 14. In addition, at least some of the cross-linking agents 14 are covalently functionalized with a plurality of functional groups 16 that protrude out of the cross-linking agents and include a charge-imparting group 17 at their distal ends.

Additional embodiments of the present disclosure pertain to methods of administering a composition of the present disclosure to a subject. In some embodiments illustrated in FIG. 1B, the methods of the present disclosure include a step of administering an active agent-containing composition to the subject (step 20) to result in the sustained release of the active agent to the subject (step 22).

As set forth in more detail herein, the methods and compositions of the present disclosure can have numerous embodiments. For instance, the compositions of the present disclosure can include various cyclic macromolecules, cross-linking agents, functional groups, and active agents. Furthermore, various methods may be utilized to administer the active agent-containing compositions of the present disclosure to a subject. Various methods may also be utilized to make the compositions of the present disclosure.

### Compositions

As set forth in more detail herein, the compositions of the present disclosure can include various cyclic macromolecules, cross-linking agents, functional groups, and active agents. For example, the compositions of the present disclosure can include a cross-linked network of cyclic macromolecules. In some embodiments, the cyclic macromolecules are covalently cross-linked to one another by a plurality of cross-linking agents. In some embodiments, at least some of the cross-linking agents are covalently functionalized with a plurality of functional groups. In some embodiments, the plurality of functional groups include a chain of at least three atoms that protrude out of the cross-linking agents.

Thus, an aspect of the present disclosure relates to a composition comprising a cross-linked network of cyclic macromolecules and a plurality of functional groups, wherein the cyclic macromolecules are covalently cross-linked to one another by a plurality of cross-linking agents, and the plurality of functional groups are covalently associated with the cross-linked network of cyclic macromolecules, wherein the plurality of functional groups comprise (i) a proximal end covalently associated with the cross-linked network of cyclic macromolecules, and (ii) a distal end protruding out of the cross-linked network of cyclic macromolecules, wherein the distal end comprises a charge-imparting group.

In any aspect or embodiment described herein, the cross-linking agents and the functional groups form a polymer network. In any aspect or embodiment described herein, the polymer network is in the form of a polymer matrix. In any aspect or embodiment described herein, the polymer network provides structural integrity to the compositions of the present disclosure. In any aspect or embodiment described herein, the polymer network includes poly (β-amino ester).

In any aspect or embodiment described herein, the composition is in the form of particles. In any aspect or embodiment described herein, the composition forms particles. For example, in any aspect or embodiment described herein, the compositions of the present disclosure form particles upon the addition of one or more (e.g., 1, 2, 3, 4, or more) active agents. In addition, the compositions of the present disclosure may have various advantageous properties.

### Composition Forms

The compositions of the present disclosure can include various forms and/or structures. Additionally, the compositions of the present disclosure may form various types of particles. For instance, in any aspect or embodiment described herein, the compositions of the present disclosure are in the form of particles that have a shell-like structure (e.g., the shell-like structure shown in FIG. 1A with an amphiphilic core and a hydrophilic outer surface).

In any aspect or embodiment described herein, the particles include an amphiphilic core (e.g., an amphiphilic core comprising a hydrophobic portion and a hydrophilic portion) and a hydrophilic outer surface. In any aspect or embodiment described herein, the hydrophilic outer surface has a positive charge. In any aspect or embodiment described herein, the hydrophilic outer surface has a negative charge. In any aspect or embodiment described herein, the hydrophilic outer surface has a neutral charge.

In any aspect or embodiment described herein, the hydrophilic outer surface has a mean surface charge of at least about −1 mV. In any aspect or embodiment described herein, the hydrophilic outer surface has a mean surface charge of at least about −3 mV. In any aspect or embodiment described herein, the hydrophilic outer surface has a mean surface charge of at least about −5 mV. In any aspect or embodiment described herein, the hydrophilic outer surface has a mean surface charge of at least about −7 mV. In any aspect or embodiment described herein, the hydrophilic outer surface has a mean surface charge of at least about −10 mV.

In any aspect or embodiment described herein, the hydrophilic outer surface has a mean surface charge ranging from about −40 mV to about 10 mV (e.g., about −40 mV to about 10 mV, about −40 mV to about 5 mV, about −40 mV to about 0 mV, about −40 mV to about −5 mV, about −40 mV to about −10 mV, about −40 mV to about −15 mV, about −40 mV to about −20 mV, about-35 mV to about 10 mV, about −35 mV to about 5 mV, about −35 mV to about 0 mV, about −35 mV to about −5 mV, about −35 mV to about −10 mV, about −35 mV to about −15 mV, about −30 mV to about 10 mV, about −30 mV to about 5 mV, about −30 mV to about 0 mV, about −30 mV to about −5 mV, about −30 mV to about −10 mV, about −25 mV to about 10 mV, about −25 mV to about 5 mV, about −25 mV to about 0 mV, about −25 mV to about −5 mV, about −20 mV to about 10 mV, about −20 mV to about 5 mV, about −20 mV to about 0 mV, about −15 mV to about 10 mV, about −15 mV to about 5 mV, or about −10 mV to about 10 mV). In any aspect or embodiment described herein, the hydrophilic outer surface has a mean surface charge ranging from about −10 mV to about 0 mV.

The particles of the present disclosure can have numerous sizes. For instance, in any aspect or embodiment described herein, the particles include diameters ranging from about 10 nm to about 10 μm. In any aspect or embodiment described herein, the particles include diameters ranging from 10 nm to about 500 nm. In any aspect or embodiment described herein, the particles include diameters ranging from 100 nm to about 500 nm. In any aspect or embodiment described herein, the particles include diameters of more than about 500 nm. In any aspect or embodiment described herein, the particles include diameters ranging from 500 nm to about 1000 nm.

In any aspect or embodiment described herein, the particles are in the form of colloidal particles. In any aspect or embodiment described herein, the particles are in the form of microparticles. In any aspect or embodiment described herein, the particles are in the form of nanoparticles.

In any aspect or embodiment described herein, the particles are in a aggregated form (e.g., a depot or depot formulation).

In any aspect or embodiment described herein, the particles are in non-aggregated form. In any aspect or embodiment described herein, the non-aggregated particles are not physically associated with one another. In any aspect or embodiment described herein, the non-aggregated particles are in dispersed form. In any aspect or embodiment described herein, the dispersed non-aggregated particles are in the form of a colloidal suspension.

### Cyclic Macromolecules

The compositions of the present disclosure can include various types of cyclic macromolecules. For instance, in any aspect or embodiment described herein, the cyclic macromolecules include, without limitation, cyclic oligosaccharides, macrocycles, cyclodextrins, and combinations thereof. In any aspect or embodiment described herein, the cyclic macromolecules include cyclodextrins. In any aspect or embodiment described herein, the cyclic macromolecules include β-cyclodextrin.

In any aspect or embodiment described herein, the cyclic macromolecules include cyclic macromolecules with at least seven membered rings. In any aspect or embodiment described herein, the cyclic macromolecules include cyclic macromolecules with at least six membered rings. In any aspect or embodiment described herein, the cyclic macromolecules include, without limitation, α-cyclodextrin, γ-cyclodextrin, and combinations thereof. In any aspect or embodiment described herein, the cyclic macromolecules include derivatives of β-cyclodextrin. For example, in any aspect or embodiment described herein, the derivatives of β-cyclodextrin include acrylated β-cyclodextrin.

### Cross-Linking Agents

The compositions of the present disclosure can include various types of cross-linking agents. For instance, in any aspect or embodiment described herein, the cross-linking agents include polyacrylic acids. In any aspect or embodiment described herein, the cross-linking agents include acrylate-based cross-linking agents. In any aspect or embodiment described herein, the cross-linking agents include, without limitation, diacrylate-based cross-linking agents (for example, alkanediol diacrylates of varying length, polyethylene glycol diacrylates of varying length, or a combination thereof). In any aspect or embodiment described herein, the cross-linking agents include polyacrylate-based cross-linking agents. In any aspect or embodiment described herein, the cross-linking agents include or is

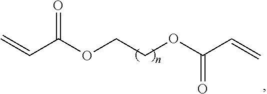

wherein n is an integer from 1 to 10 (e.g., 2, 4, 6, 8, or 10, or 1, 3, 5, 7, or 9) or 1 to 5 (e.g., 2 or 4, or 1, 3, or 5). For example, in any aspect or embodiment described herein, the cross-linking agents include or is 1,4-butanediol diacrylate, 1,6-hexanediol diacrylate, or a combination thereof. In any aspect or embodiment described herein, the cross-linking agents include acrylic-based polymers.

The compositions of the present disclosure may include various amounts of cross-linking agents. For example, in any aspect or embodiment described herein, the moles of cross-linking agents are in excess of the moles of cyclic macromolecules. By way of further example, in any aspect or embodiment described herein, the molar ratio of the cyclic macromolecules and the cross-linking agents in the composition is 1:15 to 1:50 (e.g., 1:20 to 1:50, 1:20 to 1:25, 1:22, or 1:45). For instance, in any aspect or embodiment described herein, the molar ratio of cyclic macromolecules and cross-linking agents in the composition is 1:1. In any aspect or embodiment described herein, the molar ratio of cyclic macromolecules and cross-linking agents in the composition ranges from 1:1 to 1:25. In any aspect or embodiment described herein, the molar ratio of cyclic macromolecules and cross-linking agents in the composition is 1:21, 1:22, or 1:45.

### Functional Groups

The compositions of the present disclosure can include various types of functional groups. For instance, in any aspect or embodiment described herein, the functional groups include, without limitation, polymers, alkyl chains, amine-based functional groups, or combinations thereof. In any aspect or embodiment described herein, the functional groups include polyethylene glycol. In any aspect or embodiment described herein, the functional groups include polyethylene glycol. In any aspect or embodiment described herein, the functional groups include amine-based functional groups. In any aspect or embodiment described herein, the amine-based functional groups are exposed to a surface of the particles.

In any aspect or embodiment described herein, the functional groups include polymers (e.g., hydrophilic polymers). In any aspect or embodiment described herein, the functional groups include polyethylene glycols. In any aspect or embodiment described herein, the polyethylene glycols are in the form of homopolymers.

In any aspect or embodiment described herein, the functional groups include amine-based functional groups. In any aspect or embodiment described herein, the amine-based functional groups include N,N-dimethylethylamine.

In any aspect or embodiment described herein, the functional groups include targeting agents (e.g., antibodies, peptides, small molecules, or other kinds of molecules that are able to specifically bind to an epitope or a target of interest). In any aspect or embodiment described herein, the functional groups include imaging agents For example, in any aspect or embodiment described herein, the imaging agent includes or is a fluorescent molecule, a radioactive molecule, a chelating agent, which is capable of binding a secondary imaging molecules (e.g., use of NODAGA for Cu-64 chelation that enabled positron emission tomography (PET) imaging). In any aspect or embodiment described herein, the compositions of the present disclosure lack any co-polymers. In any aspect or embodiment described herein, the compositions of the present disclosure lack polylactic acid (PLA). In any aspect or embodiment described herein, the compositions of the present disclosure lack poly (lactic-co-glycolic acid) (PLGA).

In any aspect or embodiment described herein, the functional groups include functional groups with a molecular weight of at least 100 Da. In any aspect or embodiment described herein, the functional groups include functional groups with a molecular weight of at least 200 Da. In any aspect or embodiment described herein, the functional groups include functional groups with a molecular weight of at least 300 Da. In any aspect or embodiment described herein, the functional groups include functional groups with a molecular weight of at least 400 Da. In any aspect or embodiment described herein, the functional groups include functional groups with a molecular weight of at least 500 Da.

In any aspect or embodiment described herein, the functional groups include functional groups with a molecular weight of less than 500 Da. In any aspect or embodiment described herein, the functional groups include functional groups with a molecular weight of less than 400 Da. In any aspect or embodiment described herein, the functional groups include functional groups with a molecular weight of less than 300 Da. In any aspect or embodiment described herein, the functional groups include functional groups with a molecular weight of less than 250 Da.

In any aspect or embodiment described herein, the functional groups of the present disclosure include a proximal end that is covalently associated with the cross-linked network of cyclic macromolecules and a distal end that protrudes out of the cross-linked network of cyclic macromolecules. In any aspect or embodiment described herein, the plurality of functional groups are covalently coupled directly to at least some of the cyclic macromolecules. In any aspect or embodiment described herein, the plurality of functional groups are covalently coupled directly to some or all of the cyclic macromolecules. In any aspect or embodiment described herein, the plurality of functional groups are covalently coupled directly to at least some of the cross-linking agents. In any aspect or embodiment described herein, the plurality of functional groups are covalently coupled directly to some or all of the cross-linking agents. In any aspect or embodiment described herein, the plurality of functional groups are covalently coupled directly to (i) some or all of the cyclic macromolecules, (ii) some or all of the cross-linking agents, or (iii) both some or all of the cyclic macromolecules and some or all of the cross-linking agents.

In any aspect or embodiment described herein, the distal ends of the functional groups include one or more charge-imparting groups. In any aspect or embodiment described herein, the charge-imparting groups are positioned at the distal ends of the plurality of functional groups. In any aspect or embodiment described herein, the charge-imparting groups include, without limitation, proton donating groups, proton accepting groups, positively charged groups, negatively charged groups, hydrogen donating groups, amine groups, N,N-dimethylethylamine, carboxylic acids, phosphates, sulfonates, or combinations thereof. In any aspect or embodiment described herein, the charge-imparting groups include negatively charged groups. In any aspect or embodiment described herein, the charge-imparting groups include carboxylic acids.

The functional groups of the present disclosure can include various lengths. For example, in any aspect or embodiment described herein, the plurality of functional groups include a chain of at least three (e.g., 3, 4, 5, 6, 7, 8, 9, 10, 11, 12, 13, 14, 15, 16, 17, 18, 19, 20, or more) atoms. In any aspect or embodiment described herein, the plurality of functional groups include a chain of at least ten (e.g., 10, 11, 12, 13, 14, 15, 16, 17, 18, 19, 20, or more) atoms. In any aspect or embodiment described herein, the plurality of functional groups include a chain of at least eleven (e.g., 7, 8, 9, 10, 11, 12, 13, 14, 15, 16, 17, 18, 19, 20, or more) atoms.

In any aspect or embodiment described herein, the functional groups of the present disclosure include polyethylene glycol functional groups. In any aspect or embodiment described herein, the polyethylene glycol functional groups include a chain of at least ten (e.g., 10, 11, 12, 13, 14, 15, 16, 17, 18, 19, 20, or more) atoms. For example, in any aspect or embodiment described herein, the functional group includes or is —(CH₂)ₓN(CH₃)₂, —(CH₂)ₓPO₃H—, —(CH₂)ₓCOOH, —((CH₂)₂O)ₘCH₃, —((CH₂)₂O)_{y}(CH₂)₂COOH, or a combination thereof, wherein x is an integer from 1 to 5 (e.g., 2 or 3), m is an integer from 1 to 15 (e.g., 1-12, 1, 2, 3, 4, 5, 6, 7, 8, 9, 10, 11, 12, 13, 14, or 15), and y is an integer from 1 to 5 (e.g., 2-3, 1, 2, 3, 4, or 5).

In any aspect or embodiment described herein, the molar ratio of the cross-linking agents to the amine-based functional groups in the composition is 1:0.5 to 1:1.75 to (e.g., 1:0.6 to 1:1.6, 1:0.7 to 1:1.5, 1:1.5 or 1.4:1).

### Active Agents

In any aspect or embodiment described herein, the compositions of the present disclosure can be associated with various types of active agents. In any aspect or embodiment described herein, the active agent is associated with the composition through non-covalent interactions such as, but not limited to, ionic interactions, hydrophobic interactions, hydrogen bonding interactions, and combinations thereof. In any aspect or embodiment described herein, the active agent is associated with the composition through covalent bonds. In any aspect or embodiment described herein, the active agent becomes associated with particles through interaction between the active agent and the plurality of functional groups.

In any aspect or embodiment described herein, the active agent is ionizable. In any aspect or embodiment described herein, the active agent is ionized. In any aspect or embodiment described herein, the active agent is a hydrophobic molecule. In any aspect or embodiment described herein, the active agent is a hydrophobic molecule that contains ionizable or ionized moieties.

In any aspect or embodiment described herein, the active agent is a molecule that possesses biological activity. In any aspect or embodiment described herein, the active agent includes, without limitation, small molecules, peptides, drugs, hormones, analgesics, anti-epileptics, chemotherapeutics, neuroprotective agents, anti-inflammatory agents, anti-neuro-inflammatory agents, cytotoxic agents, histone deacetylase inhibitors, proteasome inhibitors, imaging agents, targeting agents, nucleotides, therapeutic nucleic acid, deoxyribonucleic acid (DNA), ribonucleic acid (RNA), messenger RNA (mRNA), microRNA (miRNA), small interfering RNA (siRNA), short hairpin RNA (shRNA), antisense RNA, circular RNA (circRNA), ribozyme (RNAzyme), deoxyribozymes (DNAzyme), or combinations thereof.

In any aspect or embodiment described herein, the active agents include imaging agents. In any aspect or embodiment described herein, the imaging agents include dyes or radiolabels (e.g., fluorescent molecule, a radioactive molecule, or a combination thereof).

In any aspect or embodiment described herein, the active agent may be associated with the particles of the present disclosure through ionic or hydrophobic interactions. In any aspect or embodiment described herein, the active agent may be entrapped not through specific interactions with the particles of the present disclosure but by precipitation from the aqueous environment.

In any aspect or embodiment described herein, the active agents may be associated with a hydrophobic portion of the amphiphilic core of the particles of the present disclosure. In any aspect or embodiment described herein, the active agents may be associated with a hydrophilic surface of the particles of the present disclosure. In any aspect or embodiment described herein, the active agents may be associated with individual components of the particles of the present disclosure (e.g., intermediate polymer components or regions that are not on the surface or core). In any aspect or embodiment described herein, the active agents may be encapsulated by the particles of the present disclosure.

In any aspect or embodiment described herein, the active agent constitutes at least about 0.1% by weight of the composition. In any aspect or embodiment described herein, the active agent constitutes at least about 0.5% by weight of the composition. In any aspect or embodiment described herein, the active agent constitutes at least about 1% by weight of the composition. In any aspect or embodiment described herein, the active agent constitutes at least about 5% by weight of the composition. In any aspect or embodiment described herein, the active agent constitutes at least about 10% by weight of the composition. In any aspect or embodiment described herein, the active agent constitutes at least about 15% by weight of the composition. In any aspect or embodiment described herein, the active agent constitutes at least about 20% by weight of the composition. In any aspect or embodiment described herein, the active agent constitutes at least about 25% by weight of the composition. In any aspect or embodiment described herein, the active agent constitutes at least about 30% by weight of the composition. For example, in any aspect or embodiment described herein, the active agent constitutes about 0.1% to about 50% (e.g., about 0.1% to about 50%, about 0.1% to about 45%, about 0.1% to about 40%, about 0.1% to about 35%, about 0.1% to about 30%, about 0.1% to about 25%, about 0.1% to about 20%, about 0.1% to about 15%, about 0.1% to about 10%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 5% to about 50%, about 5% to about 45%, about 5% to about 40%, about 5% to about 35%, about 5% to about 30%, about 5% to about 25%, about 5% to about 20%, about 5% to about 15%, about 10% to about 50%, about 10% to about 45%, about 10% to about 40%, about 10% to about 35%, about 10% to about 30%, about 10% to about 25%, about 10% to about 20%, about 15% to about 50%, about 15% to about 45%, about 15% to about 40%, about 15% to about 35%, about 15% to about 30%, about 15% to about 25%, about 20% to about 50%, about 20% to about 45%, about 20% to about 40%, about 20% to about 35%, about 20% to about 30%, about 25% to about 50%, about 25% to about 45%, about 25% to about 40%, about 25% to about 35%, about 30% to about 50%, about 30% to about 45%, about 30% to about 40%, about 35% to about 50%, about 35% to about 45%, OR about 40% to about 50%) by weight of the composition.

The compositions of the present disclosure may be associated with various types of active agents. For instance, in any aspect or embodiment described herein, the active agents include ionizable moieties, such as hydroxamic acids. In any aspect or embodiment described herein, the active agent is a histone deacetylase inhibitor. In any aspect or embodiment described herein, the active agent can include, without limitation, panobinostat, quisinostat, dacinostat, givinostat, bortezomib, camptothecin, nile red, cytarabine, and combinations thereof.

In any aspect or embodiment described herein, the active agents of the present disclosure include drugs that are non-covalently associated with the particles of the present disclosure. In any aspect or embodiment described herein, the active agents of the present disclosure include imaging agents that are covalently bound or non-covalently associated with the particles of the present disclosure. In any aspect or embodiment described herein, the active agents of the present disclosure include targeting agents that are covalently bound to or non-covalently associated with the particles of the present disclosure.

### Methods of Administering Active Agents to a Subject

The compositions of the present disclosure may additionally be utilized to administer active agents to a subject in various manners and methods. For example, in any aspect or embodiment described herein, the method can include administering a composition of the present disclosure that is associated with an active agent to a subject. In any aspect or embodiment described herein, the administered compositions of the present disclosure can be in the form of a therapeutic formulation.

### Administration

Administration of the compositions of the present disclosure to the subject can occur through various mechanisms. For example, in any aspect or embodiment described herein, the administering occurs by a method that includes, without limitation, intravenous administration, subcutaneous administration, transdermal administration, topical administration, intraarterial administration, intrathecal administration, intradural administration, epidural administration, direct administration to subarachnoid space on the brain, administration to the cerebral ventricles or cisterna magna or other regions in close proximity to the subarachnoid space, intracranial administration, intraperitoneal administration, intraspinal administration, intranasal administration, intraocular administration, oral administration, intratumor administration, and combinations thereof.

In any aspect or embodiment described herein, the administration results in the sustained release of the active agent into a desired tissue or region of the subject.

In any aspect or embodiment described herein, administering the composition is intrathecal-cisterna magna (IT-CM) administration for delivery of the active agent to a brain (e.g., brain subarachnoid space) of the subject.

In any aspect or embodiment described herein, administering the composition is intrathecal-lumbar (IT-L) administration for delivery of the active agent to a spinal cord of the subject.

In any aspect or embodiment described herein, the compositions of the present disclosure enhance the spatial delivery of the active agents of the present disclosure to a desired region or tissue when compared to the administration of the active agent without the composition. For example, in any aspect or embodiment described herein, the compositions of the present disclosure enhance the spatial delivery of the active agents of the present disclosure to a desired region or tissue when compared to the administration of the active agent by itself. In any aspect or embodiment described herein, the compositions of the present disclosure enhance the spatial delivery of the active agents of the present disclosure to a desired region or tissue when compared to the administration of the active agents solubilized in solubilized form (e.g., an active agent solubilized in cyclodextrin or an excipient, such as cyclodextrin solubilized panobinostat).

In any aspect or embodiment described herein, the compositions of the present disclosure enhance the spatial delivery of the active agents of the present disclosure to a desired region or tissue by about 2-fold when compared to the administration of the active agent without the composition (e.g., an active agent solubilized in cyclodextrin or an excipient, such as cyclodextrin solubilized panobinostat). In any aspect or embodiment described herein, the compositions of the present disclosure enhance the spatial delivery of the active agents of the present disclosure to a desired region or tissue by about 5-fold when compared to the administration of the active agent without the composition (e.g., an active agent solubilized in cyclodextrin or an excipient, such as cyclodextrin solubilized panobinostat). In any aspect or embodiment described herein, the compositions of the present disclosure enhance the spatial delivery of the active agents of the present disclosure to a desired region or tissue by about 10-fold when compared to the administration of the active agent without the composition (e.g., an active agent solubilized in cyclodextrin or an excipient, such as cyclodextrin solubilized panobinostat). In any aspect or embodiment described herein, the compositions of the present disclosure enhance the spatial delivery of the active agents of the present disclosure to a desired region or tissue by about 20-fold when compared to the administration of the active agent without the composition (e.g., an active agent solubilized in cyclodextrin or an excipient, such as cyclodextrin solubilized panobinostat).

In a specific example, Applicant compared Panobinostat delivery between panobinostat loaded in CDN-5 (pCDN) and panobinostat solubilized in cyclodextrin (pCD). The pCD and pCDN groups were not dosed at identical levels for this example. Applicant dosed at the maximum tolerable dose only: for a single injection, this is 2 μg for pCD and 8 μg for pCDN. Thus, pCDNs were administered at 4x the dose. Applicant's results showed that the concentration in the brain compartment scaled with this dose (4×) at a single time point (2 hours). However, in the spinal cord, Applicant saw remarkably enhanced delivery (50× in the cervical region). Additionally, an increase in the volume of distribution (by 50%) was also observed. This improvement in distribution is a major aspect of various embodiments of the present disclosure.

### Release of Active Agent

The administration of the compositions of the present disclosure can result the release of the active agents from the compositions in various manners. For example, in any aspect or embodiment described herein, the active agent is released through at least 6 hours (e.g., at least 1, 2, 3, 4, 5, 6, 7, 8, 9, 10, 11, or 12 hours) after administration. In any aspect or embodiment described herein, the active agent is released through at least 1 day after administration. In any aspect or embodiment described herein, the active agent is released through at least 2 days after administration. In any aspect or embodiment described herein, the active agent is released through at least 7 days after administration. In any aspect or embodiment described herein, the active agent is released through at least 10 days after administration. In any aspect or embodiment described herein, the active agent is released through at least 14 days after administration. In any aspect or embodiment described herein, the active agent is released through at least 21 days after administration.

In any aspect or embodiment described herein, the active agent has IC₅₀ values of less than 1 μM (e.g., less than 1 μM, 0.9 μM, 0.8 μM, 0.7 μM, 0.6 μM, 0.5 μM, 0.4 μM, 0.3 μM, or 0.2 μM). In any aspect or embodiment described herein, the active agent has IC₅₀ values of less than 0.10 μM (e.g., less than 0.09 μM, 0.08 μM, 0.07 μM, or 0.06 μM). In any aspect or embodiment described herein, the active agent has IC₅₀ values of less than 0.05 μM (e.g., less than 0.04 μM, 0.03 μM, or 0.02 μM). In any aspect or embodiment described herein, the active agent has IC₅₀ values of less than 0.01μ M (e.g., less than 0.0090 μM, 0.0080 μM, 0.0070 μM, or 0.0060 μM, 0.005 μM, 0.4 μM, 0.3 μM, or 0.2 μM). In any aspect or embodiment described herein, the active agent has IC₅₀ values of less than 0.0050 μM (e.g., less than 0.0040 μM, 0.0030 μM, 0.0020 μM, or 0.0010 μM).

### Methods of Making Therapeutic Formulations

Additional embodiments of the present disclosure pertain to therapeutic formulations of the compositions disclosed herein. The therapeutic formulations of the present disclosure generally include compositions of the present disclosure that are associated with active agents. As such, a further aspect and embodiments of the present disclosure pertain to methods of making the therapeutic formulations of the present disclosure. In any aspect or embodiment described herein, such methods include associating the compositions of the present disclosure with an active agent.

Various methods may be utilized to associate the compositions of the present disclosure with an active agent. For example, in any aspect or embodiment described herein, the association occurs by doping the compositions of the present disclosure with the active agent. In any aspect or embodiment described herein, the association occurs by mixing the compositions of the present disclosure with the active agent. In any aspect or embodiment described herein, the association occurs by mechanically agitating the compositions of the present disclosure with the active agent. In any aspect or embodiment described herein, the association occurs by vortexing. In any aspect or embodiment described herein, the association occurs by incubation (e.g., passive incubation).

Association of active agents with the compositions of the present disclosure can occur through various mechanisms. For instance, in any aspect or embodiment described herein, the association occurs by self-assembly. In any aspect or embodiment described herein, the self-assembly involves complexation.

Association of active agents with the compositions of the present disclosure can occur under various conditions. For example, in any aspect or embodiment described herein, the association occurs in an aqueous medium. In any aspect or embodiment described herein, the association occurs at room temperature. In any aspect or embodiment described herein, the association occurs at temperatures below room temperature. In any aspect or embodiment described herein, the association occurs at temperatures above room temperature. In any aspect or embodiment described herein, the association occurs at a neutral pH. In any aspect or embodiment described herein, the association occurs at an acidic pH. In any aspect or embodiment described herein, the association occurs at a basic pH. In any aspect or embodiment described herein, the association occurs without the need for adjustment of any physical parameters, such as temperature and/or pH. Thereafter, the compositions of the present disclosure can be retrieved and concentrated through various processes (e.g., washing, filtration, or lyophilization).

### Applications and Advantages

The compositions and methods of the present disclosure can have various advantageous properties and applications. For instance, in any aspect or embodiment described herein, the methods of the present disclosure provide facile and one-step processes for preparing the compositions of the present disclosure through self-assembly. Moreover, in any aspect or embodiment described herein, the methods of the present disclosure do not require changes of physical parameters, such as, for example, temperature and/or pH.

Furthermore, in any aspect or embodiment described herein, the compositions of the present disclosure can be utilized to load higher amounts of active agents (e.g., 5 wt % or more in terms of active agent to composition ratio) than existing compositions. In any aspect or embodiment described herein, the compositions of the present disclosure can be utilized for the sustained release of bound active agents into various desired tissues.

Furthermore, the structures of the compositions of the present disclosure can be modified in various manners in order to accommodate the association and release of various active agents. For example, in any aspect or embodiment described herein, cross-linker length, surface charge density, the accessibility of hydrophobic portion of the amphiphilic cores, or a combination thereof, can be modified in order to maximize the loading and release of active agents.

Additionally, in any aspect or embodiment described herein, the general utility of the compositions of the present disclosure provide for an efficient drug-loading platform for hydrophobic drugs with ionizable moieties. In additional embodiments, the compositions of the present disclosure provide for architectures to enhance drug loading capacities of active agents and biophysical characteristics of the active agents.

The compositions of the present disclosure may also exhibit limited aggregation, enhanced biological stability, limited disassembly, and optimal biophysical properties for in vivo applications. In any aspect or embodiment described herein, the compositions of the present disclosure may be utilized to facilitate delivery of active agents (e.g., small molecules) into tissues. For instance, in any aspect or embodiment described herein, the compositions of the present disclosure may be utilized to deliver small hydrophobic molecules much further (e.g., spatially) into tissue when compared to other modes of administration.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

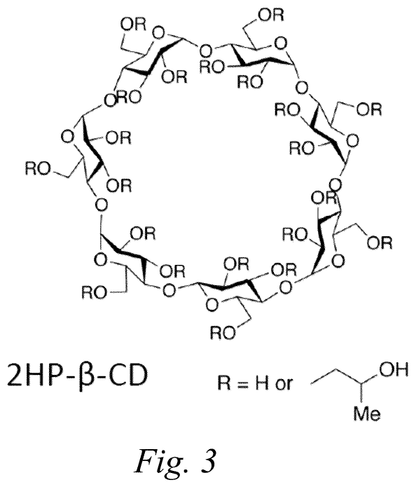

Histone deacetylase (HDAC) inhibitors arrest cell cycle and induce apoptosis. HDAC inhibitors sensitize cells to deoxyribonucleic acid (DNA) damaging therapies, by regulating chromatin contraction. Panobinostat (FIG. 2) was screened out via in vitro assays. Cyclodextrin complexation of HDAC inhibitors increases the aqueous solubility, bioavailability, and chemical stability of HDAC inhibitors, as seen with MTX110 (2-hydroxypropyl-β-cyclodextrin with Panobinostat). See FIG. 3. MTX110 is currently in Phase I clinical trials for diffuse intrinsic pontine glioma (DIPG) in children.

### Exemplary Synthesis of Cyclodextrin Nanoparticles (CDNs).

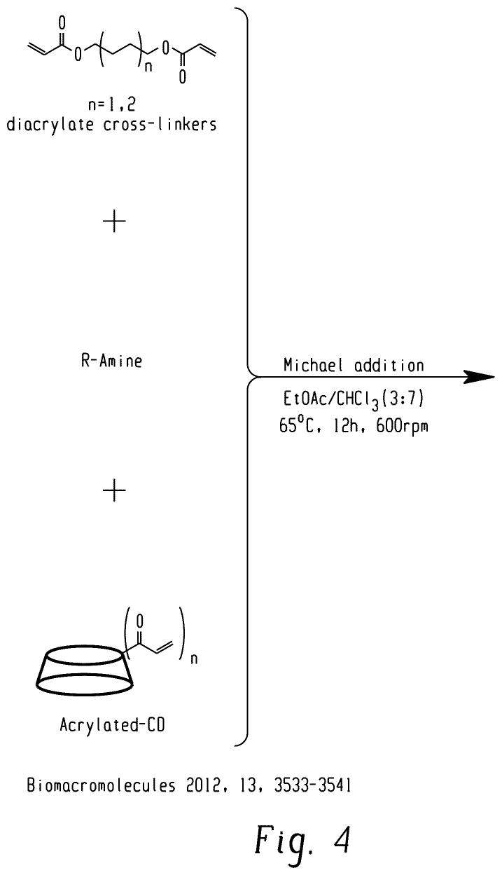

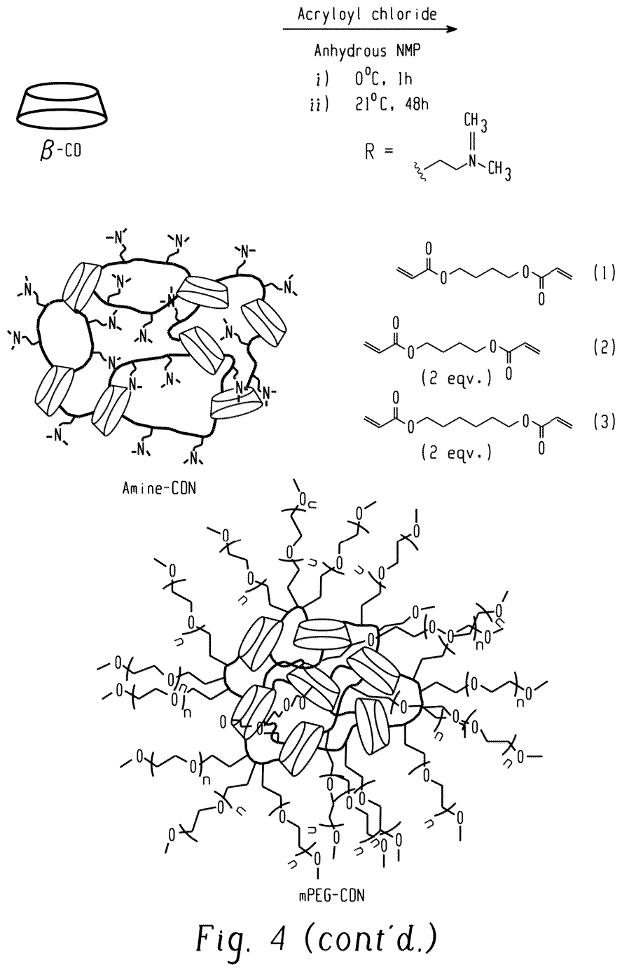

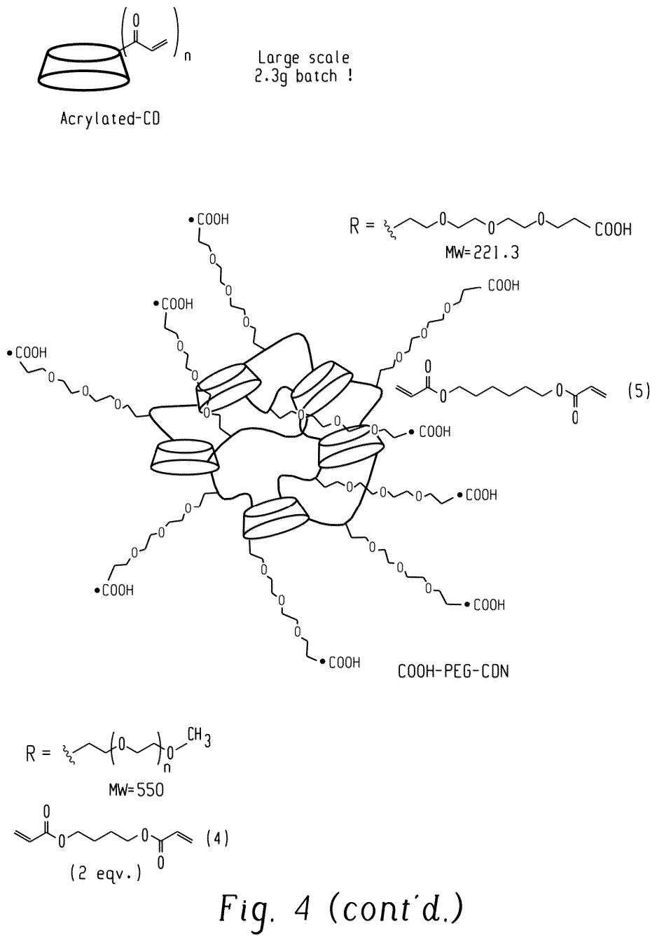

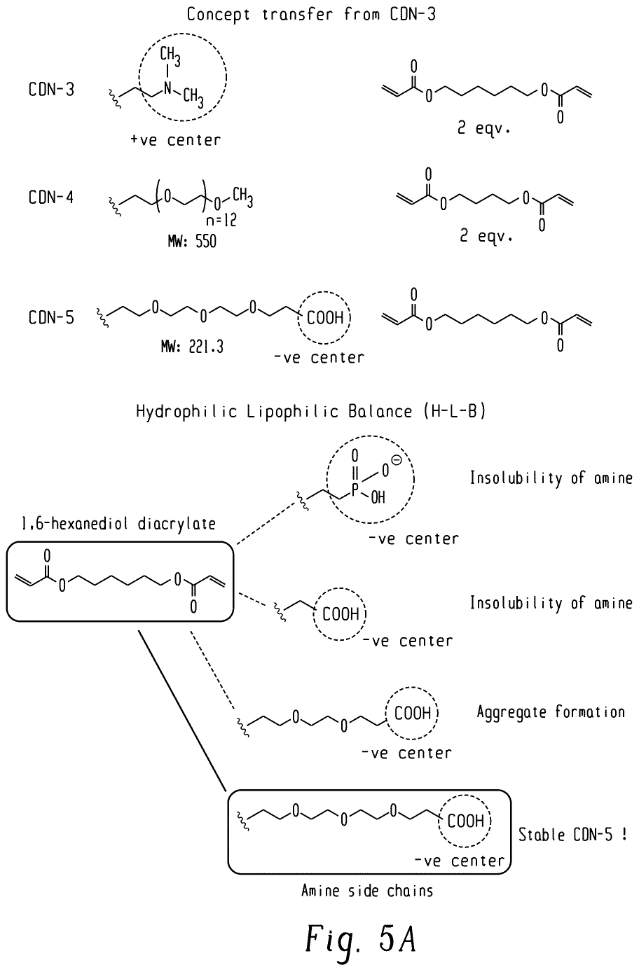

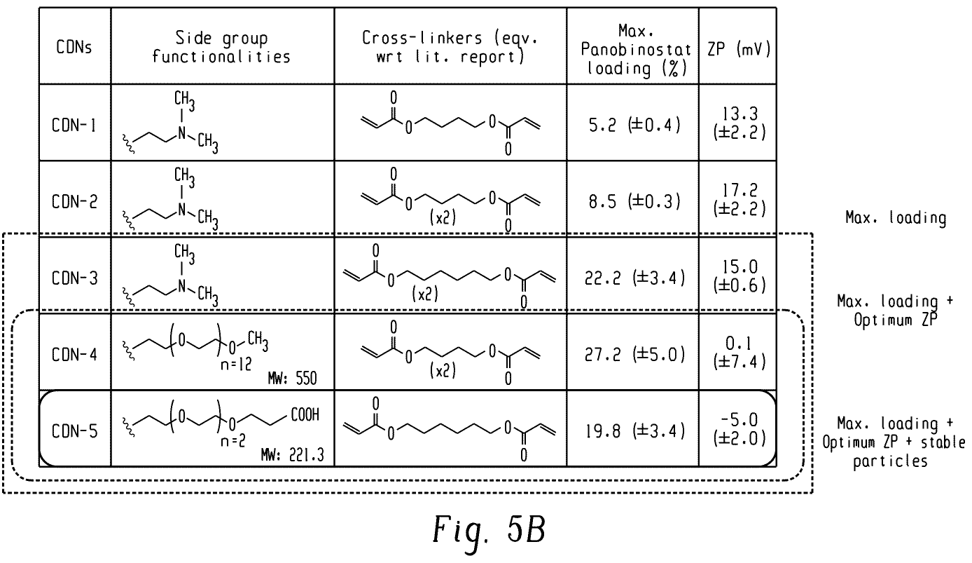

An exemplary method of synthesizing of cyclodextrin nanoparticles (CDNs) is shown in FIG. 4. Briefly, β-cyclodextran was reacted with acryloyl chloride in anhydrous N-methyl-2-pyrrolidone (NMP) at 0° C. for 1 hour and then 21° C. for 48 hours to produce acrylated-cyclodextrin (CD). Amine-cyclodextrin nanoparticles (CDNs) were prepared through Michael addition of diacrylate cross-linkers, R-Amine, wherein R is the functional group of the CDN, and acrylated-cyclodextrin (CD) with EtOAc/CHCl₃ (3:7) at 65° C. and 600 rotations per minute for 12 hours to produce CDN-1, CDN-2, CDN-3, CDN-4, and CDN-5, as shown in FIGS. 4, 5A, and 5B.

### Preparation of Exemplary Panobinostat Loaded Cyclodextrin Nanoparticles (CDNs).

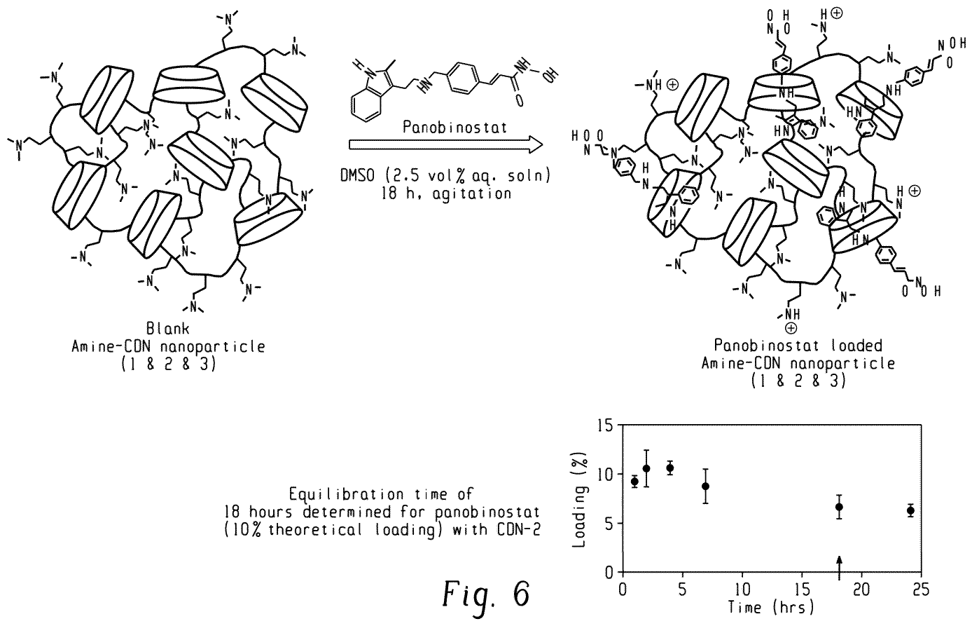

A schematic of the preparation of the Panobinostat loaded cyclodextrin nanoparticle (CDN) is shown in FIG. 6. A fresh Eppendorf vial was charged with 10 mg/mL (2.0 mL) of CDN suspension in endotoxin-free deionized water. Next, 50.0 μL of panobinostat stock solution in dimethyl sulfoxide (40 mg/mL for 10% theoretical loading) to the CDN suspension. The contents of the vial were vortexed for a few minutes, and then agitated for approximately 18 hours on a rocking platform set at speed 2. The drug (panobinostat) crashes out in the aqueous medium. The vials were centrifuged for a few seconds so that the precipitate crashed out. The supernatant was subjected to the next steps.

Half a milliliter of the suspension was applied to Amicon® Ultra-15 centrifugal filter units, 3 kDa MWCO and centrifuged for 20 minutes at a relative centrifugal force (RCF) of 5000×g at 23° C. This was performed three more times with 0.5 mL each time.

The filtrate was retrieved using a glass pipette and aliquoted into a pre-weighed Eppendorf tubes (typically about 40 μL per tube) and the rest was stored at −80° C. for future use. The aliquoted vials were lyophilized overnight. The lyophilized vials were re-suspended at 20 mg/mL concentration in endotoxin-free deionized water for dynamic light scattering (DLS) and zeta potential analysis.

### Exemplary Synthesis of Cyclodextrin Nanoparticles-5 (CDN-5).

CDN-5 particles were prepared as described above, in according to Table 1. The amount recovered (mg), size (nm), polydisparity index (PDI), and zeta potential of the 5 replicates were examined and shown in Table 1.

TABLE 1
Repeat trials for blank CDN-5 (R = COOH-PEG₃)

| | Acrylated | 1,6- | COOH- | | | Amount | Size/PDI (nm) | Zeta |
|---|---|---|---|---|---|---|---|---|
| Sl. | CD | Diacrylate | PEG₃- | | | Recovered | (post lyophilization) | Potential |
| No. | (mg) | (μL) | NH₂ (mg) | Solvent | Washing | (mg) | *PBS meas. | (mV) |
| 5.1 | 45.0 | 151.2 | 210.2 | EtOAc/CHCl₃ | UF(10k | 49.7 | | −3.6 |
| | | | | (3:7) | MWCO) | | | (±5.5) |
| | | | | 5 mL | | | | |
| 5.2 | 45.0 | 151.2 | 210.2 | EtOAc/CHCl₃ | UF(10k | 50.9 | 385.1 | −6.4 |
| | | | | (3:7) | MWCO) | | (±52.0)/0.243 | (±3.2) |
| | | | | 5 mL | | | (±0.066) | |
| 5.3 | 45.0 | 151.2 | 210.2 | EtOAc/CHCl₃ | UF(10k | 57.9 | 621.3 | |
| | | | | (3:7) | MWCO) | | (±206.3)/0.305 | |
| | | | | 5 mL | | | (±0.104) | |
| 5.4 | 45.0 | 151.2 | 210.2 | EtOAc/CHCl₃ | UF(10k | 52.3 | 538.8 | |
| | | | | (3:7) | MWCO) | | (±212.9)/0.221 | |
| | | | | 5 mL | | | (±0.026) | |
| 5.5 | 45.0 | 151.2 | 210.2 | EtOAc/CHCl₃ | UF(10k | 50.4 | 496.3 | |
| | | | | (3:7) | MWCO) | | (±185.6)/0.181 | |
| | | | | 5 mL | | | (±0.026) | |
| 5.6 | 45.0 | 151.2 | 210.2 | EtOAc/CHCl3 | UF(10k | 72.2 | | |
| | | | | (3:7) | MWCO) | | | |
| | | | | 5 mL | | | | |
| 5.7 | 45.0 | 151.2 | 210.2 | EtOAc/CHCl3 | UF(10k | 71.8 | | |
| | | | | (3:7) | MWCO) | | | |
| | | | | 5 mL | | | | |
| 5.8 | 45.0 | 151.2 | 210.2 | EtOAc/CHCl3 | UF(10k | 72.0 | | |
| | | | | (3:7) | MWCO) | | | |
| | | | | 5 mL | | | | |
| 5.9 | 45.0 | 151.2 | 210.2 | EtOAc/CHCl3 | UF(10k | 58.5 | | |
| | | | | (3:7) | MWCO) | | | |
| | | | | 5 mL | | | | |
| 5.10 | 45.0 | 151.2 | 210.2 | EtOAc/CHCl3 | UF(10k | 69.3 | | |
| | | | | (3:7) | MWCO) | | | |
| | | | | 5 mL | | | | |
| 5.11 | 45.0 | 151.2 | 210.2 | EtOAc/CHCl3 | UF(10k | 63.4 | | |
| | | | | (3:7) | MWCO) | | | |
| | | | | 5 mL | | | | |
| 5.12 | 45.0 | 151.2 | 210.2 | EtOAc/CHCl3 | UF(10k | 63.3 | | |
| | | | (BRD) | (3:7) | MWCO) | | | |
| | | | | 5 mL | | | | |
| 5.13 | 45.0 | 151.2 | 210.2 | EtOAc/CHCl3 | UF(10k | 60.7 | | |
| | | | (BRD) | (3:7) | MWCO) | | | |
| | | | | 5 mL | | | | |
| 5.14 | 45.0 | 151.2 | 210.2 | EtOAc/CHCl3 | UF(10k | 60.5 | | |
| | | | (BRD) | (3:7) | MWCO) | | | |
| | | | | 5 mL | | | | |
| 5.15 | 45.0 | 151.2 | 210.2 | EtOAc/CHCl3 | UF(10k | 62.7 | | |
| | | | (BRD) | (3:7) | MWCO) | | | |
| | | | | 5 mL | | | | |

### Preparation of Exemplary Panobinostat Loaded CDN-5.

CDN-5 particles were loaded with panobinostat (Pb-CDN-5) as described above with theoretical loading of 30%, 40%, and 50%. For each theoretical loading, the actual percent loading, size (nm), polydisparity index (PDI), and zeta potential of the examined Ph-CDN-5 was examined and shown in Table 2.

TABLE 2
Characteristics of exemplary panobinostat loaded CDN-5 (Pb-CDN-5)

| | Theoretical | Theoretical | Theoretical |
|---|---|---|---|
| | loading (%) = 30.0 | loading (%) = 40.0 | loading (%) = 50.0 |
| | Size(nm)/PDI/ | Size(nm)/PDI/ | Size(nm)/PDI/ |
| Sl. | ZP(mV) | ZP(mV) | ZP(mV) |
| No. | *PBS meas. | *PBS meas. | *PBS meas. |
| 5.1 | | 23.7% | |
| | | 328.8 (±4.0)/0.268 | |
| | | (±0.019)/ | |
| | | −6.5 (±8.2) | |
| 5.2 | | 18.1% | 13.4% |
| | | 388.9 (±4.7)/0.298 | 384.4 (±29.8)/0.314 |
| | | (±0.020)/ | (±0.013) |
| | | −9.3 (±11.6) | |
| 5.3 | | 17.5% | |
| | | Not enough sample | |
| | | left for DLS | |
| | | and ZP | |
| 5.4 | | | 15.6% |
| | | | 387.4 (±71.5)/0.23 |
| | | | (±0.082) |
| 5.5 | 12.5% | 15.3% | |
| | Not enough sample | 293.4 (±23.9)/0.218 | |
| | left for DLS | (±0.024)/ | |
| | and ZP | | |

### Exemplary Determination of Panobinostat Loaded in Cyclodextrin Nanoparticles (CDNs).

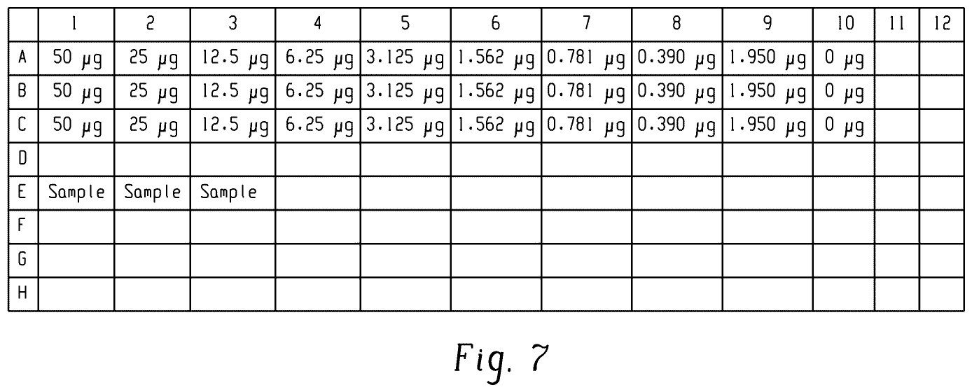

The standard calibration curve was prepared as follows. A 5 mg/mL solution of sample and control blank cyclodextrin nanoparticles (CDNs) in dimethyl sulfoxide were prepared. Nine half-dilutions in dimethyl sulfoxide were prepared starting with the stock 5 mg/mL panobinostat solution. Next, 40 μL of the 5 mg/mL control CDN solution (no drug) was added to wells A1-C10 to the plate, and each dilution (10 μL) was added to the corresponding wells to provide the standard curve in triplicate, as shown in FIG. 7. The plate was well mixed.

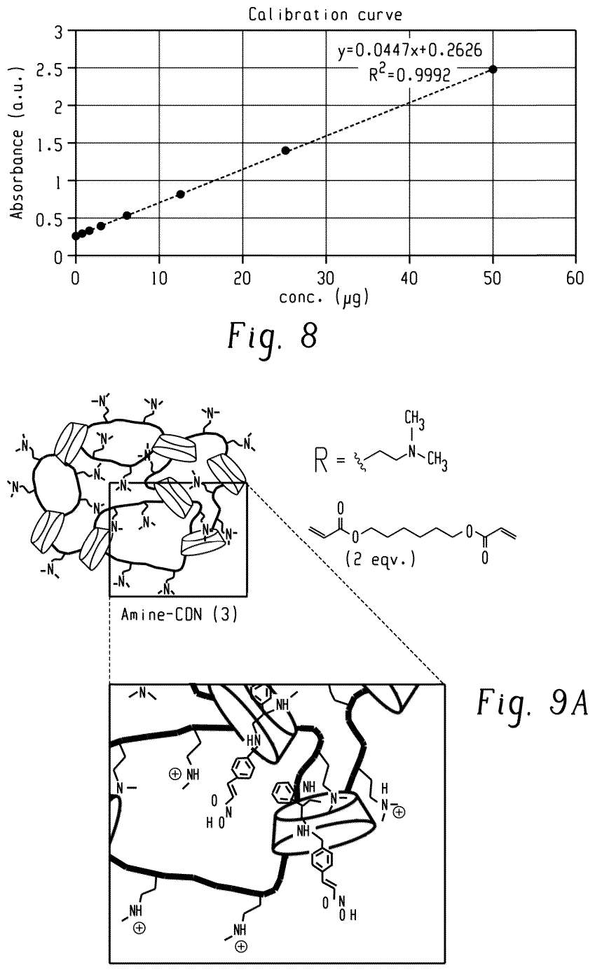

The panobinostat nanoparticle samples were run in triplicate-40 μL per well with 10 μL dimethyl sulfoxide (see, e.g., FIG. 7). The plate was swirled for approximately 10 seconds to mix the contents of the wells. The plate was read at an absorbance of 310 nm with a plate spectrophotometer. The readings for each of the triplicate concentration of the standards for the calibration curve was averaged, and a calibration curve of the drug quantity versus the average reading was produced, which is shown as FIG. 8. The equation of the line was determined to be y=0.0447x+0.2626 with an R² of 0.9992. Thus, for samples run on the same plate as this exemplary standard calibration curve, the quantity of drug loaded in the samples (ug) is equal to (Average Sample Absorbance-0.2626)/0.0447=x μg. The percentage of drug loading is equal to (x/200)*100, since 40 μL of 5 mg/mL of drug loaded CDN sample is used.

### Examination of the Stability of Exemplary Panobinostat Loaded Cyclodextrin Nanoparticles.

Drug loaded particles were freshly prepared under (A) acidic (pH=4.0), (B) neutral (pH=7.4), or (C) alkaline conditions (pH=10.2). Samples were incubated in dialysis cassettes maintained at room temperature, and nanoparticle samples were removed at regular intervals for dynamic light scattering (DLS) analysis.

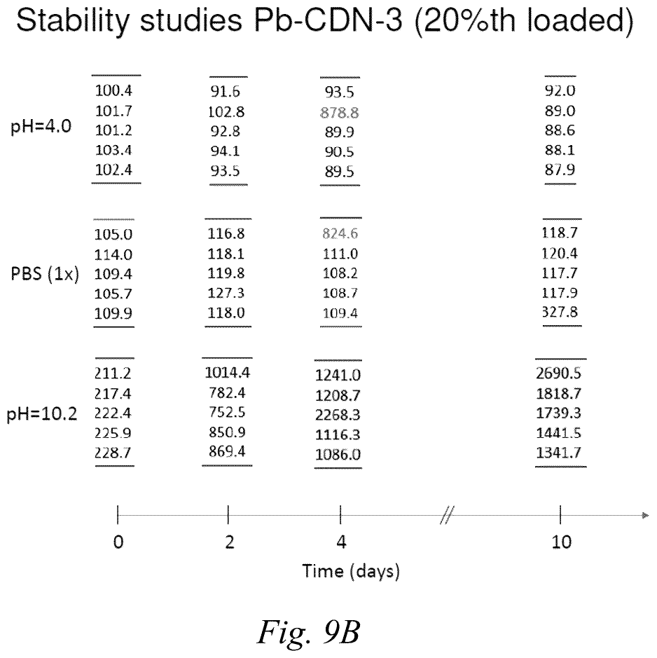

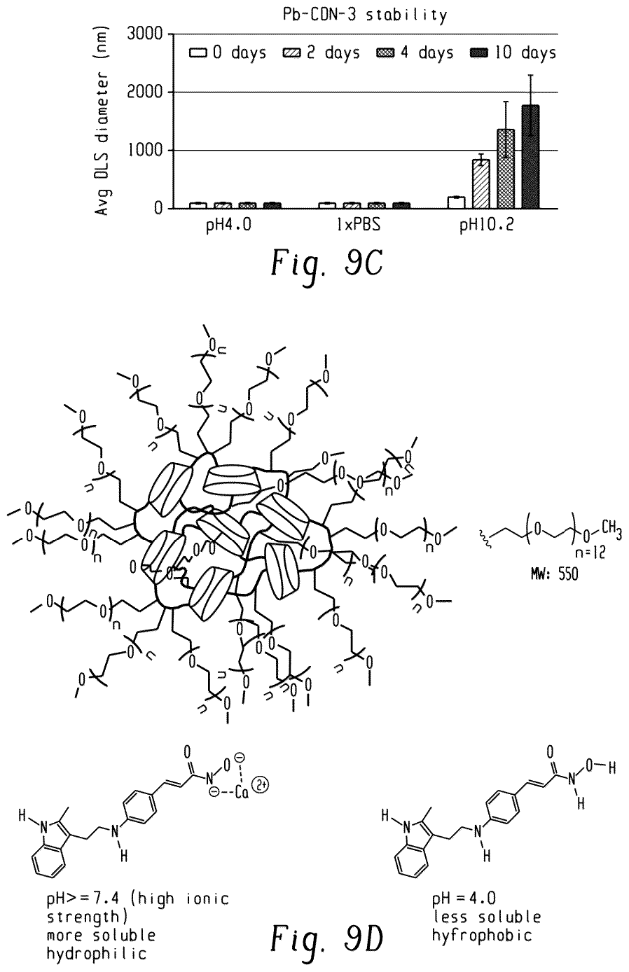

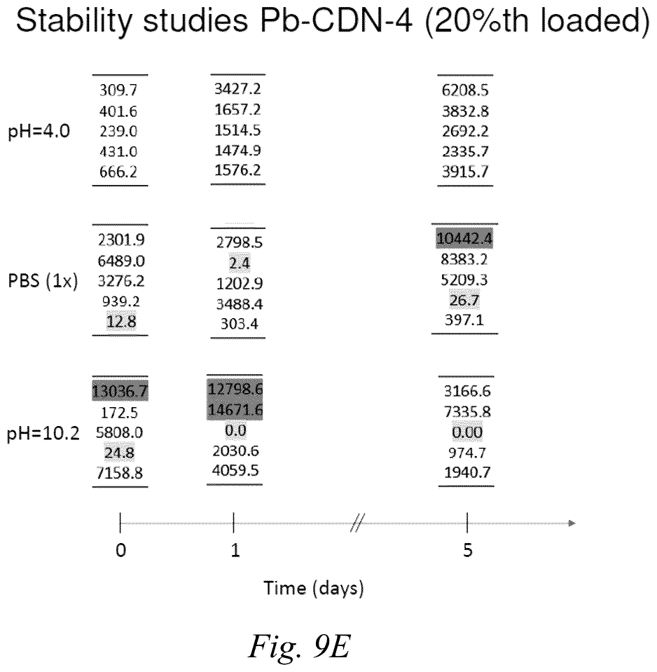

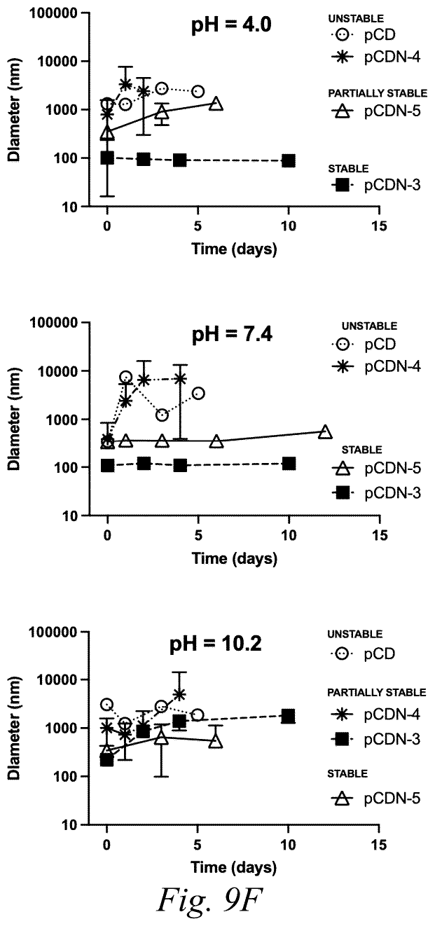

The stability of panobinostat loaded CDN-3 (FIG. 9A) and panobinostat loaded CDN-4 (FIG. 9D) were examined via dynamic light scattering (DLS) at pH 4 and 10.2, as well as in 1× phosphate-buffered saline. FIG. 9B and FIG. 9C show the observed stability of panobinostat loaded CDN-3 (Pb-CDN-3) at days 0, 2, 4, and 10. FIG. 9E shows the observed stability of panobinostat loaded CDN-4 (Pb-CDN-4) at days 0, 1, and 5.

The data shown in FIG. 9F, with all values represent mean plus/minus standard deviation, was measured in triplicate and data describing samples for which aggregation was observed is represented by a dashed line. pCD and pCDN-4 aggregated in aCSF regardless of pH. pCDN-3 was highly stable in both acidic and neutral pH, with moderate instability in alkaline pH over time. pCDN-5 exhibited good stability in neutral and alkaline pH, with mild aggregation observed at later time points in acidic pH. Aggregation was only observed for pCDN-5 at the latest time point (day 6), at which point >50% of drug is expected to be released and the system will disassemble. Thus, CDN-5 nanoparticles have enhanced in vitro stability relative to alternative formulations.

### Examination of the Stability of Exemplary Panobinostat Loaded CDN-5 (Pb-CDN-5).

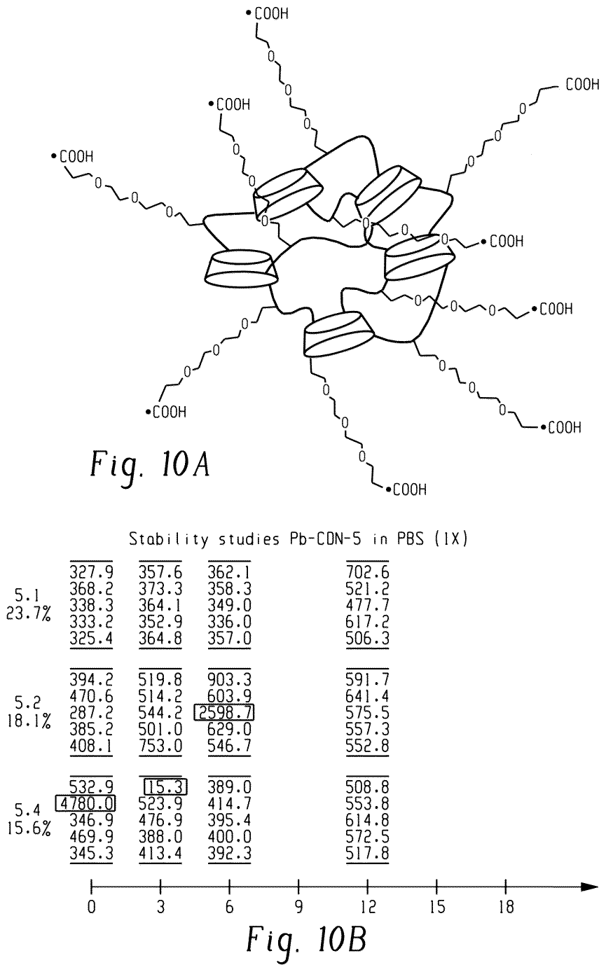

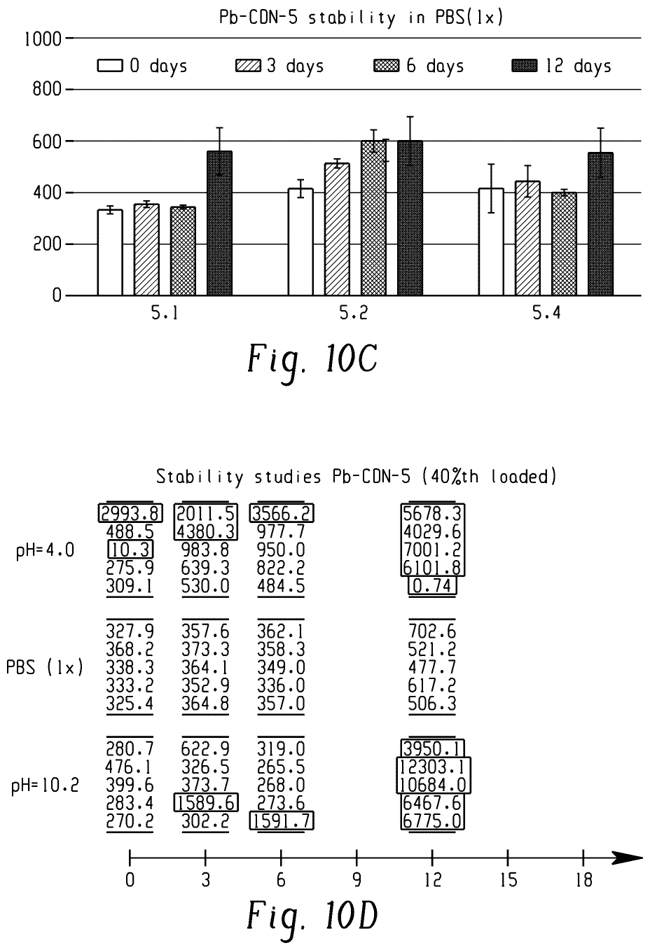

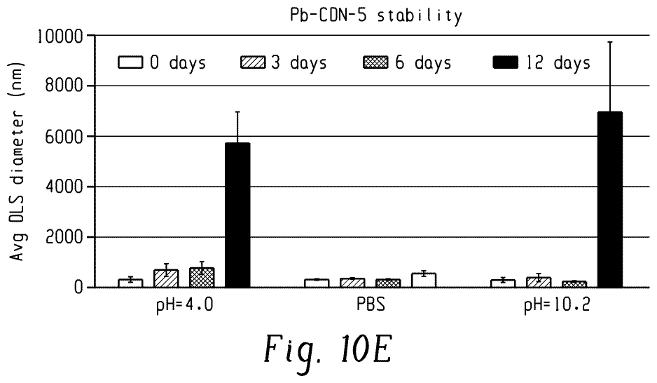

The stability of exemplary panobinostat loaded CDN-5 (Pb-CDN-5) were examined in 1× phosphate-buffered saline via dynamic light scattering (DLS). FIG. 10A illustrates the structure of CDN-5, while FIG. 10B and FIG. 10C show the observed stability of panobinostat loaded CDN-5 (Pb-CDN-5) at days 0, 3, 6, and 12 for 5.1 with 23.7% loading, 5.2 with 18.1% loading, and 5.4 with 15.6% loading. FIG. 10D and FIG. 10E show the observed stability of 40% th loaded panobinostat CDN-5 (Pb-CDN-5) for 5.5 at days 0, 3, 6, and 12.

### In Vivo Examination of Exemplary Panobinostat Loaded CDN-5 (Pb-CDN-5).

Improved dosing/tolerability for panobinostat was observed when administered via CDN-5. Freshly CDN nanoparticles loaded with Panobinostat or cyclodextrin-solubilized Panobinostat was administered by either intratumor or intrathecal-cisterna magna routes. Intratumoral administration consisted of up to 10 μL of fluid administered at a rate of <1 μL/minute. Intrathecal administration consisted of up to 20 μL of fluid administered in a fast bolus of under 30 seconds. Lack of tolerability is defined as greater than 20% weight loss or presence of neurological symptoms. For example, intratumoral infusion of panobinostat loaded CDN-4 was able to deliver dosages from 1 μg to 30 μg (30-fold), single intrathecal administration of panobinostat loaded CDN-5 was tolerated at dosages from 2 μg to 8 μg (4-fold), and multi-dose (twice weekly) intrathecal administration of panobinostat loaded CDN-5 was tolerated at dosages of 0.5 μg to 7 μg (14-fold).

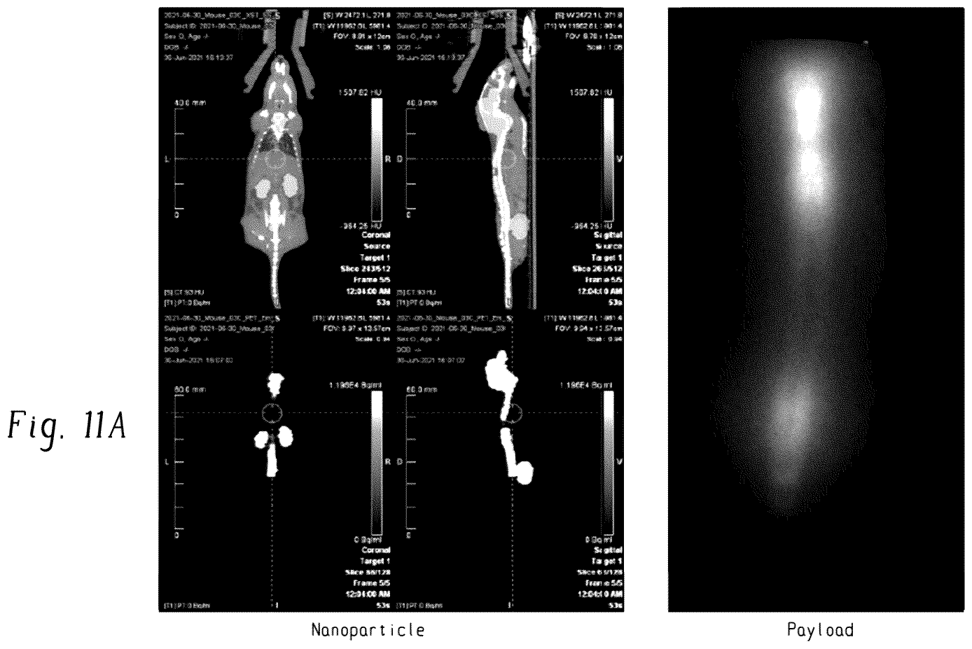

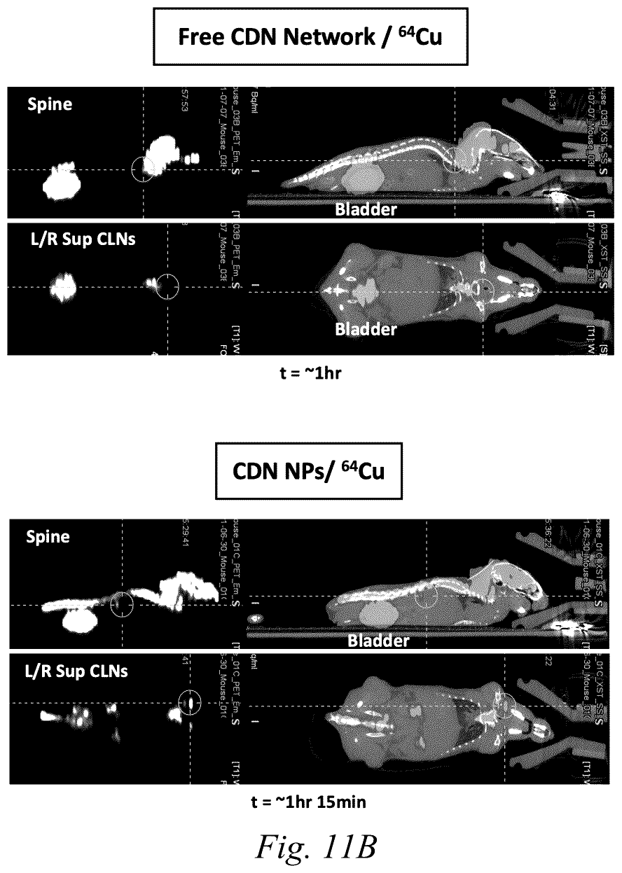

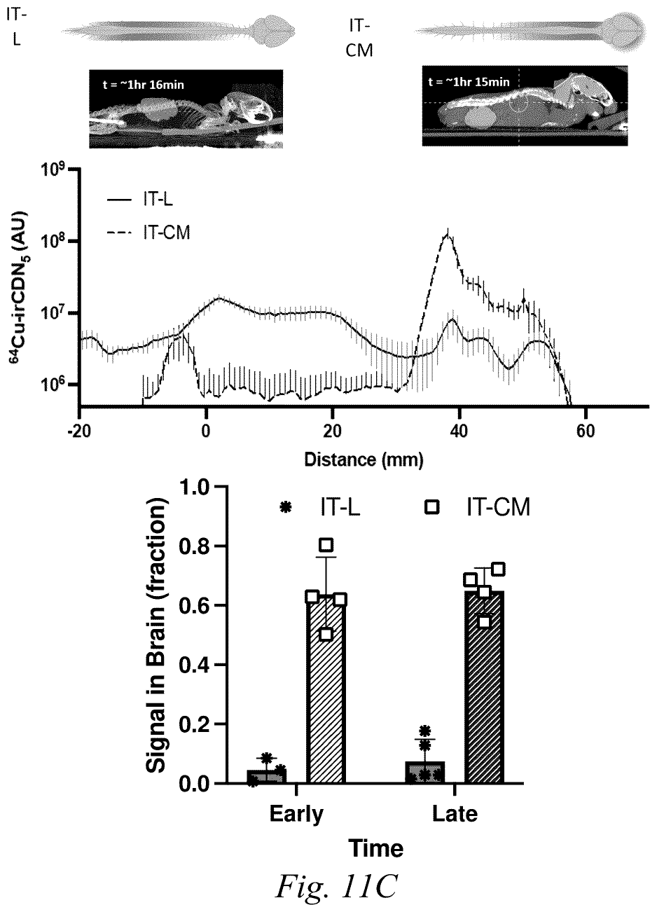

Particle distribution was examined with particles radiolabeled with Cu-64, utilizing a 1,4,7-triazacyclononane, 1-glutaric acid-4,7-acetic acid (NODA-GA) chelator on the surface of the nanoparticle. The radiolabeled nanoparticles were administered to healthy C57BL6 female mice (7-9 weeks of age), which were subsequently imaged with positron emission tomography (PET) to evaluate particle distribution. FIG. 11A demonstrates that the exemplary panobinostat loaded CDN-5 nanoparticles are able to target the spinal cord.

The assembled particles transited the subarachnoid space (lower panel of FIG. 11B), while the non-assembles free CDN network did not transit the subarachnoid space (upper panel of FIG. 11B). Thus, FIG. 11B demonstrates that particles must be assembled to transit the subarachnoid space. As shown in FIG. 11C, when ^{64Cu}CDN5 nanoparticles were administered intrathecally via the lumbar route (IT-L), delivery remained concentrated near the injection site, and nanoparticles achieved minimal access to the subarachnoid space of the brain. In contrast to the intrathecal-lumbar (IT-L) route, intrathecal-cisterna magna (IT-CM) administered nanoparticles are visualized within the subarachnoid space in both the brain and the spinal cord. Thus, FIG. 11C illustrates that IT-CM (intrathecal-cisterna magna) administration is a preferred route of administration over IT-L (intrathecal-lumbar) for achieving delivery to the brain subarachnoid space, and IT-L (intrathecal-lumbar) administration is a preferred route of administration over IT-CM (intrathecal-cisterna magna) for achieving delivery to the spinal cord.

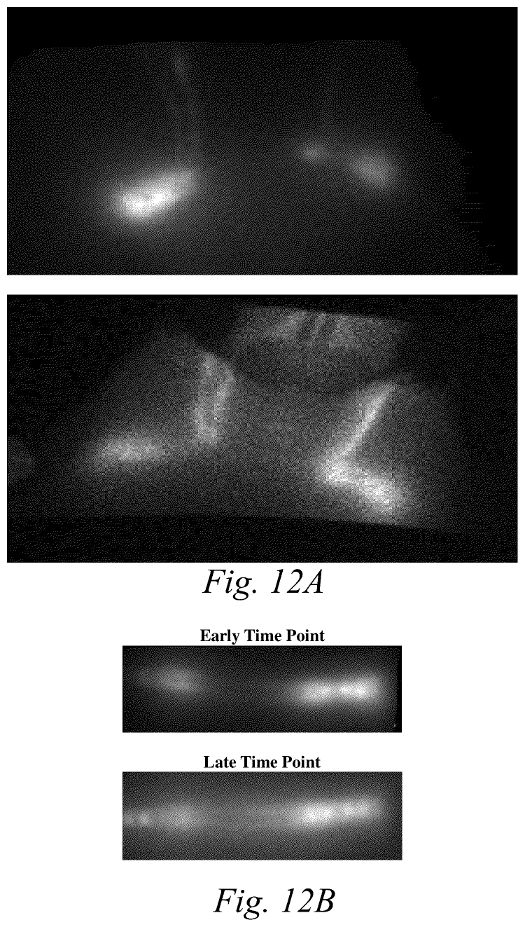

The CDN-5 nanoparticles were loaded with IR780, a near-infrared fluorescent molecule. The IR780 loaded nanoparticles were administered to healthy C57BL6 mice (n=3-5, female mice, 7-9 weeks of age), and a specialized near-infrared camera was used to evaluate the distribution of the molecule along the spinal axis and in the deep cervical lymph nodes. FIG. 12A shows representative images of payload delivery to cervical lymphatics following administration of IR780-loaded CDNs into the cisterna magna of healthy mice. FIG. 12B shows representative images of payload (IR780) delivery to the neuroaxis following administration of IR780-loaded CDNs into the cisterna magna of healthy mice at an early time point (top) and late time point (bottom). FIGS. 12A and 12B show that IR780 amount and distribution within the spinal cord are improved by encapsulation in the CDN nanoparticle.

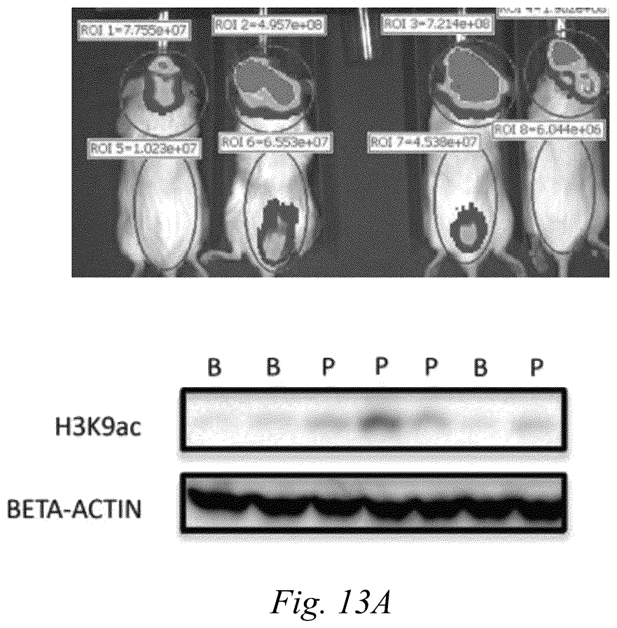

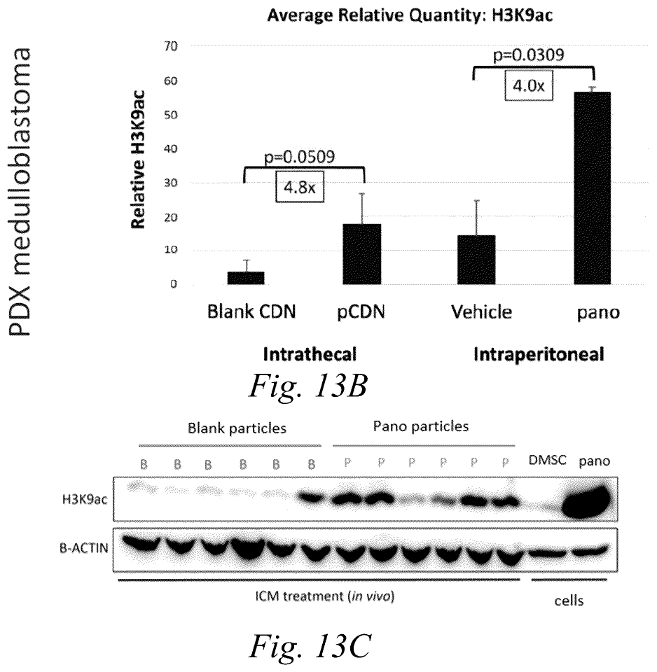

FIGS. 13A, 13B, 13C, and 13D demonstrate that the payload (panobinostat) activates pharmacodynamic targets, thereby resulting in increased levels of acetylation of lysine 9 on histone H3 (H3K9ac), when administered intrathecally or intraperitoneally.

Examining and Comparing Cyclodextrin Solubilized Panobinostat (pCD) to Panobinostat Loaded Cyclodextrin Nanoparticles (pCDN).

Overall, it was observed that higher drug exposure in panobinostat loaded cyclodextrin nanoparticles (pCDN) yielded efficacy.

TABLE 3
Quantitative analyses confirm dose-scaled tissue exposures and
improved spatial distribution

| | | Panobinostat |
|---|---|---|
| | Cyclodextrin | Loaded |
| | Solubilized | Cyclodextrin |
| | Panobinostat | Nanoparticles |
| Region | (pCD) | (pCDN) |
| Brain | 1.5 ± 1.4 μM | 6.3 ± 7.1 μM |
| Ventricle | 15 ± 10 μM | 60 ± 72 μM |

### Examination of In Vivo Drug Delivery.

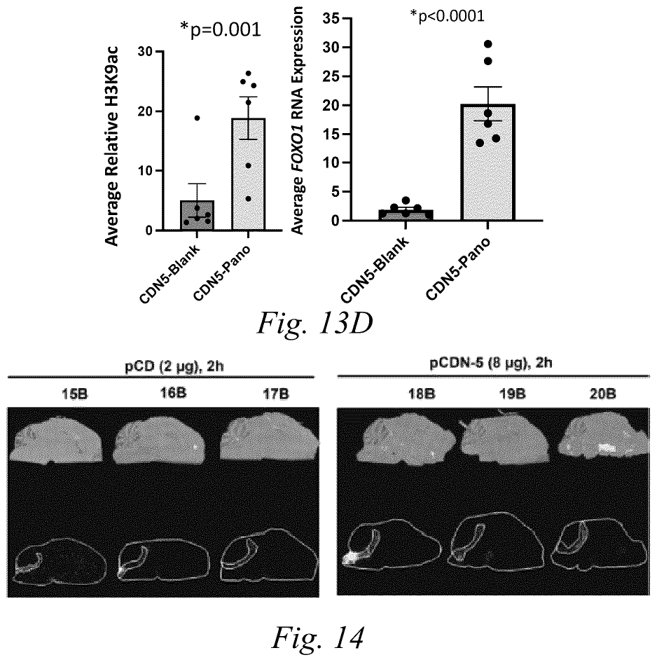

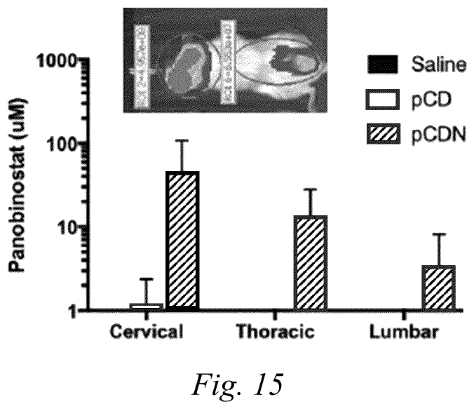

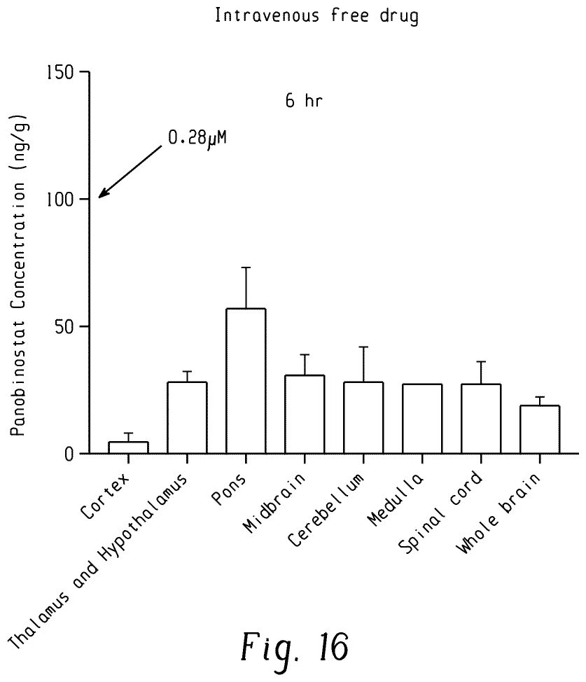

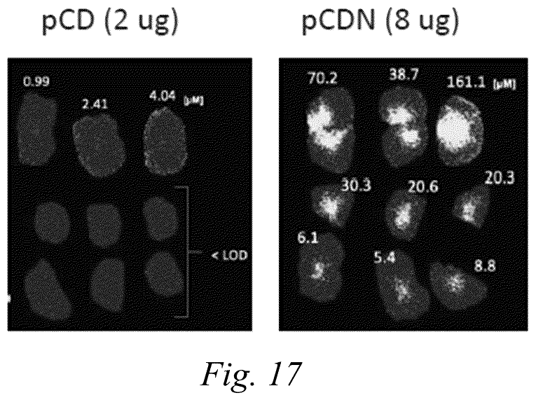

Cyclodextrin nanoparticles loaded with 8 μg of panobinostat (pCDN) or 2 μg of panobinostat solubilized with cyclodextrin (pCD) were administered into the cisterna magna of healthy C57BL/6 mice or immunodeficient NOD scid gamma (NSG) mice. The brains and whole spinal cords were extracted, frozen, and sliced into sections for matrix-assisted laser desorption/ionization mass spectrometry imaging (MALDI-MSI) analyses (representative examples are provided for the brain and spinal cord in FIG. 14 and FIG. 17, respectively). These data are shown quantitatively in FIGS. 15, 18, and 19.

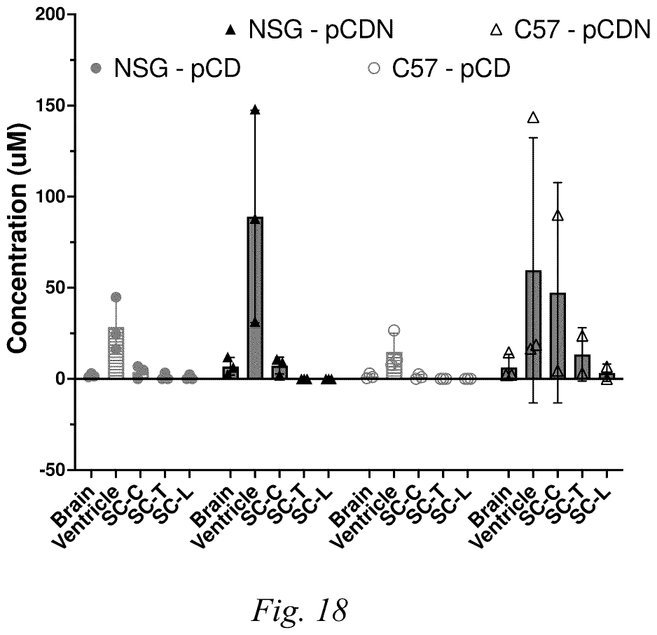

FIG. 18 shows the concentration of panobinostat after 2 hours for the entire brain (“brain”), the peri-ventricular tissue (“ventricular”), cervical spinal cord (“SC-C”), thoracic spinal cord (“SC-T”), and the lumbar spinal cord (“SC-L”). FIG. 18 demonstrates that pCDNs enhance drug delivery relative to pCDs in healthy mice.

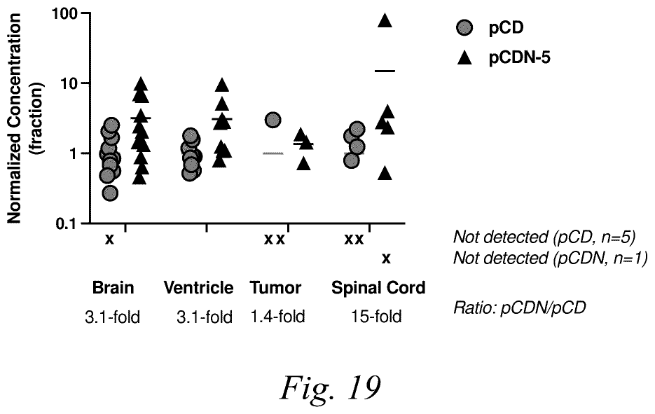

Cyclodextrin nanoparticles loaded with 8 μg of panobinostat (pCDN) or 2 μg of panobinostat solubilized with cyclodextrin (pCD) were administered into the cisterna magna of NOD scid gamma (NSG) mice bearing advanced stage, orthotopic, patient derived medulloblastoma tumors (n=3 subjects per group, female). The brains and whole spinal cords were extracted, frozen, and sliced into sections for Matrix-assisted laser desorption/ionization mass spectrometry imaging (MALDI-MSI) analyses. FIG. 19 shows the normalized concentration of panobinostat in the entire brain (“brain”), peri-ventricular tissue (“ventricle”), the tumor as identified on serial hematoxylin and eosin (H&E) stained sections (tumor), and a representative section of the spinal cord containing tumor as identified by hematoxylin and eosin (H&E) staining (“spinal cord”). “X” refers to “not detected” (i.e., 0, which cannot be plotted on a log scale). FIG. 19 demonstrates that pCDNs enhance delivery relative to pCDs in tumor-bearing mice.

### Treatment of Medulloblastoma Xenografts.

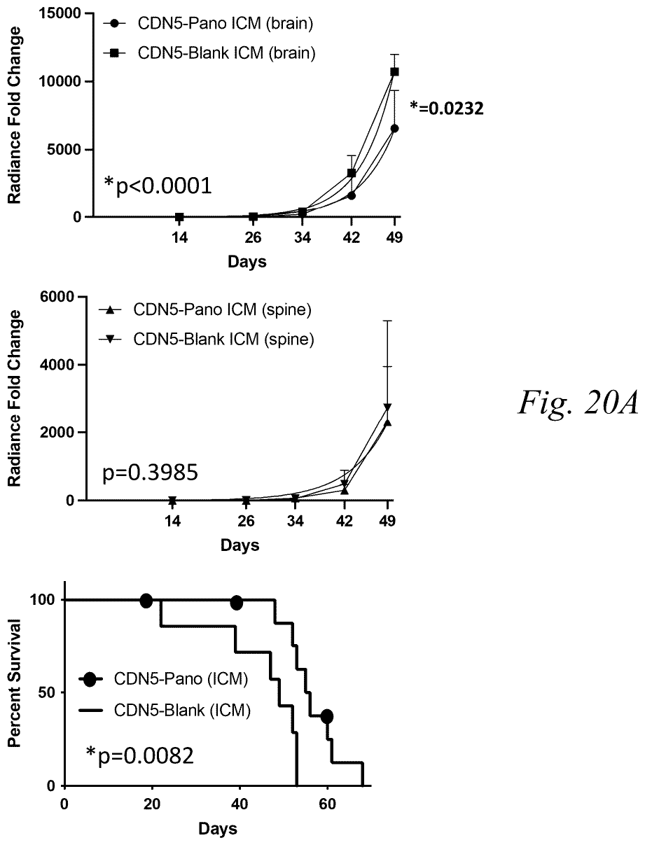

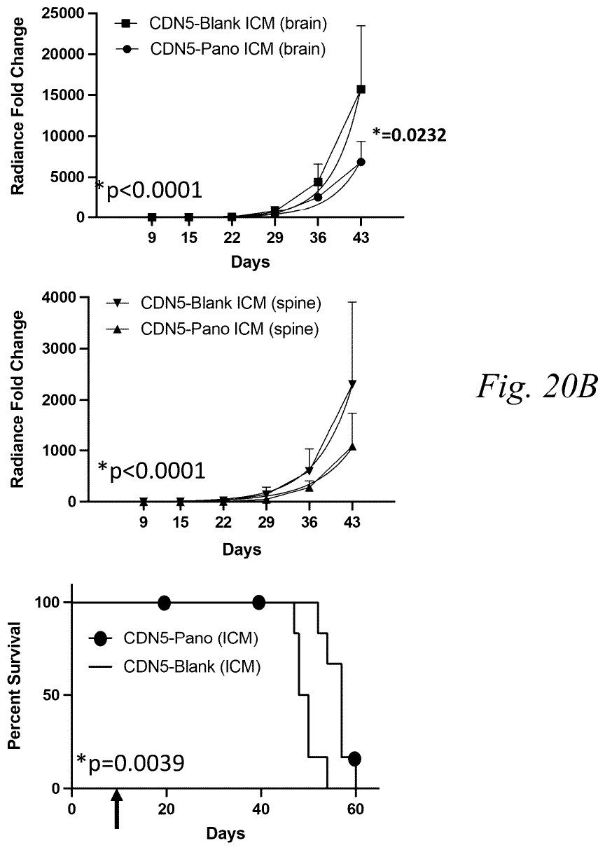

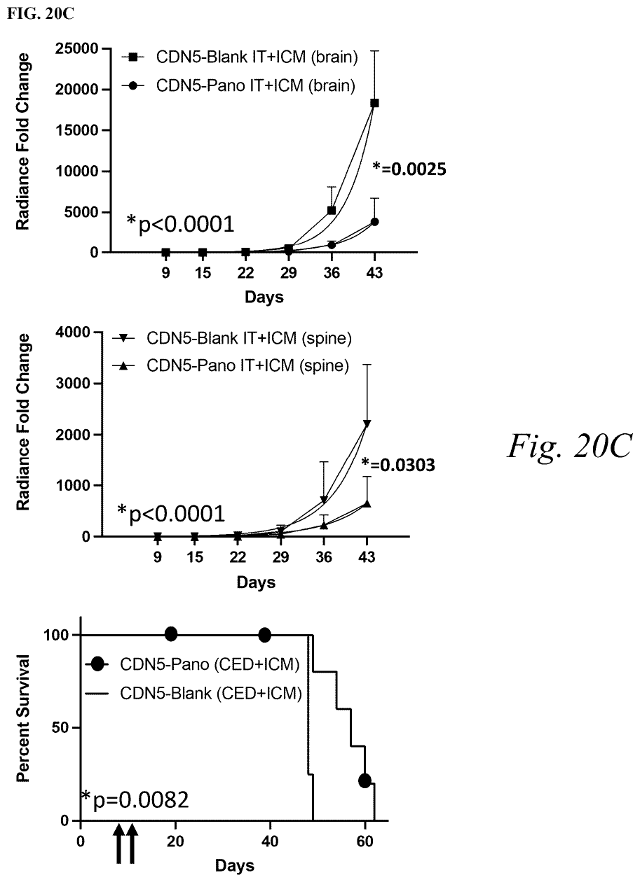

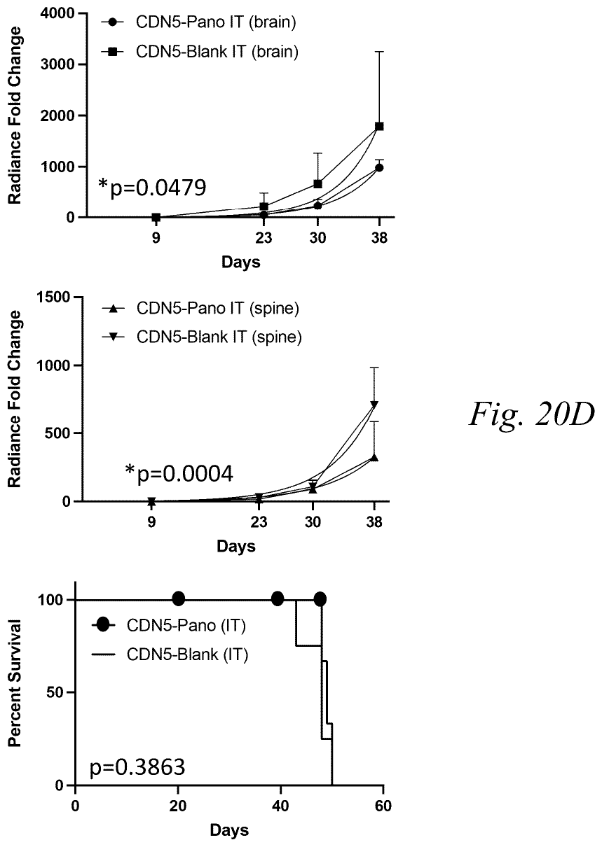

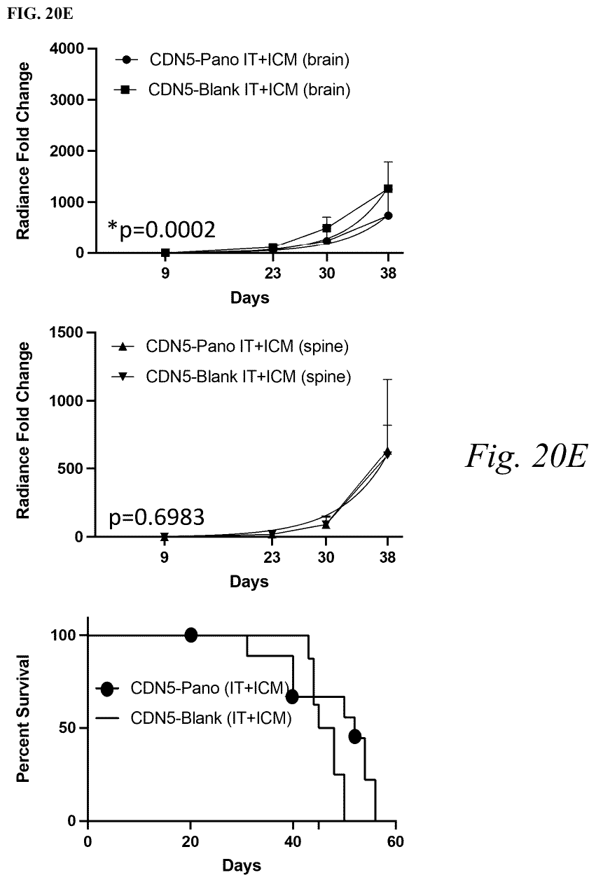

Female NOD scid gamma (NSG) mice (8-10 weeks of age) bearing orthotopic, patient derived medulloblastoma xenografts were treated with various doses of panobinostat loaded in CDN-5 (pCDN-5) or non-drug loaded CDN-5 (i.e., blank CDN-5 or CDNs) through cisterna magna (ICM) or intratumoral (IT) administration. Three replicates were performed in cohorts of n=6-8 mice each, with FIG. 20A showing the data from Experiment 1, FIGS. 20B and 20C showing the data from Experiment 2, and FIGS. 20D and 20E showing the data from Experiment 3.

FIG. 20A was performed with an intratumoral (IT) administration with 6 μg panobinostat loaded in CDN-5 (pCDN-5) or blank CDN-5. FIG. 20B was performed with an cisterna magna (ICM) administration with 6 μg panobinostat loaded in CDN-5 (pCDN-5) or blank CDN-5. FIG. 20C was performed with an cisterna magna (ICM) administration with 6 μg panobinostat loaded in CDN-5 (pCDN-5) and an intratumoral (IT) administration with 6 ug panobinostat loaded in CDN-5 (pCDN-5) or cisterna magna (ICM) and an intratumoral (IT) administration blank CDN-5. FIG. 20D was performed with an intratumoral (IT) administration with 6 μg panobinostat loaded in CDN-5 (pCDN-5) or blank CDN-5. FIG. 20E was performed with an intratumoral (IT) administration with 30 μg panobinostat loaded in CDN-5 (pCDN-5) and an cisterna magna (ICM) administration with 6 μg panobinostat loaded in CDN-5 (pCDN-5) or an intratumoral (IT) and an cisterna magna (ICM) administration blank CDN-5.

Of importance, in some instances, a primary treatment effect in the brain was observed, but not in the spinal cord. The presentation of these tumors is highly variable in terms of metastasis, and the studies were not adequately powered to handle type II (false negative) error. The analysis of the data from FIGS. 20A through 20E is shown in Table 4.

TABLE 4
Analysis of data from FIGS. 20A-20E

| | P < 0.05, | P < 0.05, | P < 0.05, | ICM | |
|---|---|---|---|---|---|
| Expt | brain? | spinal cord? | survival? | Dose | IT Dose |
| 1 | yes | (no) | yes | 6 ug | |
| 2.1 | yes | yes | yes | 6 ug | |
| 2.2 | yes | yes | yes | 6 ug | 6 ug |
| 3.1 | yes | yes | no | 6 ug | 30 ug |
| 3.2 | yes | (no) | p = 0.0685 | 6 ug | 30 ug |

Across the three biological replicates performed, the treatment of tumor bearing mice with panobinostat loaded in CDN-5 (pCDN-5) slowed the growth of orthotopic xenografts to produce statistically significant, prolonged survival.

Examination of Panobinostat Release from Panobinostat Loaded in CDN-5 (pCDN-5)

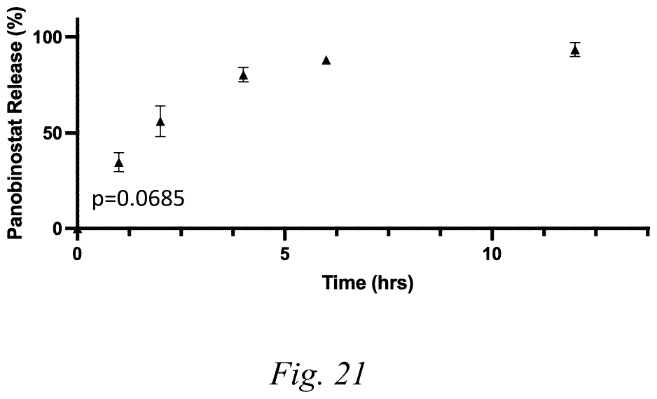

FIG. 21 shows the percent release of panobinostat from panobinostat loaded in CDN-5 (pCDN-5). Drug loading was determined by dissolving lyophilized formulation aliquots in dimethyl sulfoxide (DMSO) (5 mg/mL) and reading absorbance (310 nm) on a Tecan plate reader. Arbitrary units were converted to mass through comparison to a carefully constructed control curve, whereby known concentrations of Panobinostat were spiked into control (non-drug containing) CDN solutions (10 μL drug into 40 μL of CDN polymer in DMSO). To measure controlled release, lyophilized formulations were resuspended in water to achieve a drug concentration of ~1 mg/mL, and this solution (400 μL) was added to a 3.5 kDa MWCO Slide-A-Lyzer dialysis cassette (Thermo Fisher, Waltham, Massachusetts). Cassettes were immersed in a large volume of phosphate-buffered saline (PBS) (4 L) at 37° C. with gentle stirring (75 rotations per minute). To maintain sink conditions, release media was fully exchanged every 24 hours. A sample (30 μL) was removed at regular intervals and dissolved in 120 μL of DMSO for reading absorbance on a Tecan plate reader (Männedorf, Switzerland). Results are reported for a minimum of three separate readings for all characterization experiments.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A composition comprising:
a cross-linked network of cyclic macromolecules,
wherein the cyclic macromolecules are covalently cross-linked to one another by a plurality of cross-linking agents;
a plurality of functional groups,
wherein the plurality of functional groups are covalently associated with the cross-linked network of cyclic macromolecules,
wherein the plurality of functional groups comprise:
a proximal end covalently associated with the cross-linked network of cyclic macromolecules, and
a distal end protruding out of the cross-linked network of cyclic macromolecules,
wherein the distal end comprises a charge-imparting group.

2. The composition of claim 1, wherein the charge-imparting group is positioned at the distal ends of the plurality of functional groups.

3. The composition of claim 1 or 2, wherein the charge-imparting group is selected from the group consisting of proton donating groups, proton accepting groups, positively charged groups, negatively charged groups, hydrogen donating groups, amine groups, N,N-dimethylethylamine, carboxylic acids, phosphates, sulfonates, and combinations thereof.

4. The composition of any one of claims 1-3, wherein the charge-imparting group is a negatively charged group.

5. The composition of any one of claims 1-4, wherein the charge-imparting group comprises carboxylic acids.

6. The composition of any one of claims 1-5, wherein the plurality of functional groups are covalently coupled directly to at least some of the cyclic macromolecules.

7. The composition of any one of claims 1-6, wherein the plurality of functional groups are covalently coupled directly to at least some of the cross-linking agents.

8. The composition of any one of claims 1-7, wherein the plurality of functional groups comprises functional groups with a molecular weight of less than 500 Da or a molecular weight of less than 300 Da.

9. The composition of any one of claims 1-8, wherein the composition is in the form of particles.

10. The composition of claim 9, wherein the particles are in aggregated form or non-aggregated form.

11. The composition of claim 9 or 10, wherein the particles comprise an amphiphilic core and a hydrophilic outer surface, wherein the hydrophilic outer surface has a negative or neutral charge.

12. The composition of any one of claims 9-11, wherein the particles comprise diameters ranging from 100 nm to about 500 nm.

13. The composition of any one of claims 1-12, wherein:
the molar ratio of the cyclic macromolecules and the cross-linking agents in the composition is 1:15 to 1:50 (e.g., 1:20 to 1:50, 1:20 to 1:25, 1:22, or 1:45);
the molar ratio of the cross-linking agents to the amine-based functional groups in the composition is 1:0.5 to 1:1.75 to (e.g., 1:0.6 to 1:1.6, 1:0.7 to 1:1.5, 1:1.5 or 1.4:1);
the cyclic macromolecules are selected from the group consisting of cyclic oligosaccharides, macrocycles, cyclodextrins, and combinations thereof;
the cross-linking agents comprise polyacrylic acids; or
a combination thereof.

14. The composition of any one of claims 1-13, wherein the cyclic macromolecules comprise β-cyclodextrin.

15. The composition of any one of claims 1-14, wherein the functional groups comprise or are polymers, polyethylene glycol, alkyl chains, amine-based functional groups, or a combination thereof.

16. The composition of any one of claims 1-15, wherein
the functional groups comprise amine-based functional groups, wherein the amine-based functional groups are exposed to a surface of the particles;
the functional groups comprise polyethylene glycol functional groups; or
a combination thereof.

17. The composition of any one of claim 16, wherein the functional groups comprise polyethylene glycol functional groups that comprise a chain of at least ten atoms.

18. The composition of any one of claims 1-17, wherein the cross-linking agents and the functional groups form a polymer network.

19. The composition of claim 18, wherein the polymer network comprises poly (β-amino ester).

20. The composition of any one of claims 1-19, further comprising an active agent.

21. The composition of claim 20, wherein:
the active agent is associated with the composition through non-covalent interactions;
the active agent is ionized;
the active agent is a hydrophobic molecule;
the active agent constitutes at least about 0.1% by weight of the composition; or
a combination thereof.

22. The composition of claim 20 or 21, wherein the active agent includes or is a small molecule, peptide, drug, hormone, analgesic, anti-epileptic, chemotherapeutic, neuroprotective agent, anti-inflammatory agent, anti-neuro-inflammatory agent, cytotoxic agent, histone deacetylase inhibitor, proteasome inhibitor, imaging agent, targeting agent, nucleotides, therapeutic nucleic acid, deoxyribonucleic acid (DNA), ribonucleic acid (RNA), messenger RNA (mRNA), microRNA (miRNA), small interfering RNA (siRNA), short hairpin RNA (shRNA), antisense RNA, circular RNA (circRNA), ribozyme (RNAzyme), deoxyribozymes (DNAzyme), or combinations thereof.

23. A method of administering an active agent to a subject, wherein the method comprises:
administering a composition of any of claims 1-22 to the subject,
wherein the composition is associated with the active agent.

24. The method of claim 23, wherein:
the active agent is associated with the composition through non-covalent interactions;
the active agent includes or is a small molecule, peptide, drug, hormone, analgesic, anti-epileptic, chemotherapeutic, neuroprotective agent, anti-inflammatory agent, anti-neuro-inflammatory agent, cytotoxic agent, histone deacetylase inhibitor, proteasome inhibitor, imaging agent, targeting agent, nucleotides, therapeutic nucleic acid, deoxyribonucleic acid (DNA), ribonucleic acid (RNA), messenger RNA (mRNA), microRNA (miRNA), small interfering RNA (siRNA), short hairpin RNA (shRNA), antisense RNA, circular RNA (circRNA), ribozyme (RNAzyme), deoxyribozymes (DNAzyme), or combinations thereof;
the active agent has IC₅₀ values of less than <1.0 μM; or
a combination thereof.

25. The method of claim 23 or 24, wherein the composition enhances the spatial delivery of the active agent to a desired region or tissue when compared to the administration of the active agent without the composition.

26. The method of any one of claims 23-25, wherein:
administering the composition is intrathecal-cisterna magna (IT-CM) administration for delivery of the active agent to a brain (e.g., brain subarachnoid space) of the subject;
administering the composition is intrathecal-lumbar (IT-L) administration for delivery of the active agent to a spinal cord of the subject; or
a combination thereof.
